(19) United States
(12) Patent Application Publication
(10) Pub. No.: US 2021/0061687 A1
(43) Pub. Date: Mar. 4, 2021

(54) FUNCTIONALIZATION OF ELECTRODES WITH ELECTRICIGENIC MICROORGANISMS AND USES THEREOF

(71) Applicant: Board of Trustees of Michigan State University, East Lansing, MI (US)

(72) Inventors: Gemma Reguera, East Lansing, MI (US); Rebecca J. Steidl, Pleasant Hill, IA (US)

(21) Appl. No.: 16/993,774

(22) Filed: Aug. 14, 2020

Related U.S. Application Data
(60) Provisional application No. 62/891,574, filed on Aug. 26, 2019.

(51) Int. Cl. C02F 3/00; H01M 8/16; C25B 11/04; C02F 3/34; C12N 1/20

(52) U.S. Cl. CPC C02F 3/005; H01M 8/16; C02F 2101/34; C02F 3/341; C12N 1/20; C25B 11/0447

(57) ABSTRACT

Methods of making improved bioelectrodes are provided capable of functionalizing electrode materials in less than 24 hours. Pre-seeding the electrode with electricigenic microbes forms an electricigenic biofilm on the electrode to maximize surface coverage by electricigenic microbes and reduce the number of fastidious organisms. The method results in bioelectrodes that are functionalized in less than 24 hours and that can generate higher yields of current from a wide range of substrates. Methods for treating wastewater by removing fermentative inhibitors and generating current are provided. The bioelectrodes generated are tailored to efficiently oxidize substrates, such as the fermentative inhibitors, in the wastewater targeted for removal from the wastewater. Improved electrodes are generated rapidly with high coulombic efficiency. Bioelectrochemical systems (BESs) containing the improved electrodes are also provided.

The present application claims benefit under 35 U.S.C. 119(e) of U.S. Provisional Application Ser. No. 62/891,574, filed on Aug. 26, 2019.

### BACKGROUND

The increased concern for the inevitable depletion of the oil supply as well as the negative impact of the use of fossil fuels on the environment has highlighted the need for biofuel alternatives such as ethanol, diesel, butanol, hydrogen, and electricity produced from renewable resources. Wastewater can be generated in a number of processes such as in bioethanoi refineries, residential households, food industry, and biodiesel plants. Disposal and/or remediation of wastewater can be hazardous, time-consuming and expensive. Environmentally responsible treatment of wastewater remains a challenge. Wastewater treatment currently accounts for about 3% of energy consumption in the US alone.

### SUMMARY

The embodiments described herein include novel methods for functionalizing electrodes for use in bioelectrochemical systems. The bioelectrochemical systems can include microbial electrochemical cells, including microbial fuel cells and microbial electrolysis cells.

In one embodiment, a method of making an electrode for use in a bioelectrochemical system is provided. The method can functionalize the electrode for transfer of electrons between an electricigenic film on the surface of the electrode and the electrode in less than 24 hours. In one embodiment, the method comprises poising the electrode at a potential for metabolism of selected substrates by one or more electricigenic bacteria. In one embodiment, the metabolism can be oxidative metabolism. In one embodiment, the electrode is located in a chamber containing a liquid, such as media, water and/or wastewater. In one embodiment, the liquid in the chamber is in substantial quantities so the electrode is substantially or fully immersed so all or substantially all of the electrode surface is accessible to the cells for pre-seeding.

The method also includes, in one embodiment, pre-seeding the electrode in the chamber with an inoculum of electricigenic bacteria. In one embodiment, the electricigenic bacteria in the inoculum are grown in one or more growth mediums (i.e., growth media). In one embodiment, the growth media may comprise selected substrates added to acclimate the electricigenic bacteria for growth on the substrates. Substrates can be electron donors or the like. Substrates can be, for example, fermentative inhibitors such as lactate, pyruvate and acetate. In one embodiment, the growth media for the preparation of the inoculum can also include electron acceptors such as fumarate. In one embodiment, the substrates included in the inoculation media are the same as the substrates present in the liquid in the chamber. These substrates are targeted for removal from the liquid by degradation as a result of oxidative metabolism by the electricigenic bacteria with concomitant generation of current. The electricigenic bacteria from the inoculum attach to the electrode to form an electricigenic biofilm on the surface of the electrode. Growth of this biofilm functionalizes the electrode to efficiently metabolize the substrates in the liquid to be treated and generate current with improved coulombic efficiency.

In one embodiment, the method further includes adding supplements to the liquid in the chamber for optimizing growth of the electricigenic bacteria. In embodiments described herein, supplements include nutrients, vitamins, minerals, salts, buffering systems, nitrogen sources and combinations thereof. In one embodiment, one or more of the supplements are added to the liquid in the chamber.

In one embodiment, the electricigenic bacteria comprises an electricity-producing microorganism, i.e. an electricigen such as Geobacter sulfurreducens, (Gsu). In some embodiments, the electricigenic bacteria can include Gsu strains adaptively evolved for traits of interest. In one embodiment, the Gsu strain is alcohol-tolerant Gsu (GsuA). In one embodiment, the strains are adapted and/or genetically engineered Gsu strains such as GR51 and GR52 capable of transferring substantially all the electrons in the fermentation byproducts (e.g., hydrogen, one or more organic acids, or a combination thereof) to the anode electrode to produce electricity. In one embodiment, the Gsu strain is adaptively evolved to metabolize or more efficiently metabolize a substrate that can be metabolized by fastidious organisms in order to favor the growth of the electricigen over the fastidious organisms.

In one embodiment, the liquid is wastewater such as glycerin-containing wastewater or wastewater containing fermentative inhibitors such as lactate and acetate. In one embodiment, the formation of the electricigenic biofilm on the electrode does not require autoclaved media or sterile conditions. In one embodiment, the electrode is functionalized in less than 24 hours. In one embodiment, the electrode is functionalized in less than 12 hours. In one embodiment, the method prevents or reduces the growth of fastidious organisms resulting in improved coulombic efficiency.

In one embodiment, functionalized electrodes made according to the methods described herein are incorporated into bioelectrochemical systems or cells such as a microbial electrolysis cell or a microbial fuel cell. The functionalized electrode can be used in bioelectrochemical systems for treating wastewater such as glycerin-containing water, therein, in the presence of electricigenic biofilm. Any of the electricigen noted above may be used herein as the electricigenic bacteria. In one embodiment, the bioelectrochemical cell is a single chamber cell. In one embodiment, the bioelectrochemical cell is a H-type two-chamber cell.

In one embodiment, a method for treating wastewater is provided. The method includes providing, for example, a first electrode in a chamber with wastewater, wherein the first electrode has been functionalized by a method comprising poising the electrode at a potential optimal for oxidative metabolism of selected substrates by one or more electricigenic bacteria, pre-seeding the first electrode with an inoculum of electricigenic bacteria, wherein the electricigenic bacteria in the inoculum have been grown in growth media comprising the substrates, wherein the substrates are the metabolic targets by the electricigenic bacteria in the wastewater and allowing the electricigenic bacteria to attach to the electrode to form an electricigenic biofilm. In various embodiments, the method can further include providing a second electrode and providing a reference electrode.

In one embodiment, functionalized electrodes are provided with improved coulombic efficiency. The functionalized electrodes can comprise an electricigenic biofilm having a thickness of 0.5 μm or greater. In one embodiment, the electrode is functionalized in less than 24 hours. In one embodiment, the electrode is functionalized in less than 12 hours, or less than 8 hours, or less than 6 hours. In one embodiment, the electrode is functionalized between about one hour and about 6 hours.

In one embodiment, the electrode generates a current density of at least about 50 μA/cm² in less than 24 hours. In one embodiment, the electrode generates a current density of at least about 50 μA/cm² in less than 12 hours. In one embodiment, the electrode generates a current density of at least about 50 μA/cm² in less than 8 hours.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a simplified schematic of a method of functionalizing an electrode according to an embodiment.

FIG. 2 is a simplified schematic of a bioelectrochemical system according to an embodiment.

FIG. 3A is a schematic of a microbial electrolysis cell (MEC) according to an embodiment.

FIG. 3B is a schematic of a microbial electrolysis cell (MEC) according to an embodiment.

FIG. 3C is a schematic of a single chamber MEC according to an embodiment.

FIG. 4 is a simplified schematic of a consolidated process for ethanol and electricity generation according to an embodiment.

FIG. 5A is a graph of current production in H-type MECs using standard laboratory “DB” medium as described herein with 0.5 mM lactate-10 mM acetate (temperature 35° C.). The two lines are two independent runs to show technical biological variability.

FIG. 5B is a graph of current production in H-type MECs using condensate medium (“CM”) (temperature 35° C.). The two lines are two independent runs to show technical biological variability.

FIG. 6 is a graph of current production and acetate/lactate removal in a 2-L SCMEC driven by strain GR52 in CM (temperature 35° C.).

FIG. 7 is a graph of current production and acetate/lactate removal in a 2-L SCMEC driven by a hybrid GR52:GR51 bioelectrode in CM (temperature 35° C.).

### DESCRIPTION OF THE EMBODIMENTS

In the following detailed description, the, embodiments are described in sufficient detail to enable those skilled in the art to practice them, and it is to be understood that other embodiments may be utilized and that chemical and procedural changes may be made without departing from the spirit and scope of the present subject matter. The following detailed description is, therefore, not to be taken in a limiting sense, and the scope of the embodiments is defined only by the appended claims.

In one embodiment, a method of making an electrode for use in a bioelectrochemical system, such as a microbial electrochemical cell (MEC), by generating an electricigenic biofilm is provided. The method can functionalize electrodes for transfer of electrons between the electricigenic film on the electrode surface and the electrode. The electrode can be functionalized by establishing the electricigenic biofilm that can generate a current in less than 24 hours and in some embodiments less than 12 hours. In one embodiment, the method can include poising the electrode at a potential for metabolism of selected substrates by one or more electricigenic bacteria. In one embodiment, the method can include pre-seeding the electrode with an inoculum of electricigenic bacteria to form the biofilm. The inoculum of electricigenic bacteria used for pre-seeding can be generated by culturing the desired electricigenic bacteria in media supplemented with selected substrates and/or electron acceptors. In one embodiment, the electricigenic bacterial culture can be centrifuged and the cells in the pellet can then be used as the inoculum for pre-seeding of the electrode in order to form the desired electricigenic biofilm. In one embodiment, the growth of the electricigenic biofilm and the functionalization of the electrode may not require sterile media, a sterile environment and/or sterile conditions. In one embodiment, the functionalized electrode can generate current within 24 hours of the addition of the pre-seeding microbes.

In one embodiment, a Geobacter microbe can serve as the electricigen. In one embodiment, Geobacter sulfurreducens (Gsu) may serve as the electricigen. In one embodiment, one or more Gsu strains with adaptively evolved traits of interest may be used. In one embodiment, one or more Gsu strains are genetically engineered and adaptively evolved for selective utilization of carbon sources, e.g. as GsuA, GR51, GR52 and the like may be used to form the electricigenic biofilm.

In one embodiment, a method of treating wastewater is provided. The method can include using a functionalized electrode in a bioelectrochemical system having an electricigenic biofilm specifically directed to oxidatively metabolize the carbon sources present in the wastewater to be treated. In one embodiment, substrates in the wastewater include acetate, pyruvate and lactate that can serve as electron donors to the electricigenic biofilm. In one embodiment, the removal of fermentative inhibitors such as acetate and lactate from the wastewater can be accomplished efficiently by the incorporation of a functionalized electrode as described herein in a bioelectrochemical system. In one embodiment, a Geobacter microbe can serve as the electricigen. In one embodiment, Geobacter sulfurreducens (Gsu) can serve as the electricigen. In one embodiment, one or more Gsu strains genetically engineered and/or adaptively evolved for selective utilization of carbon sources and/or alcohol tolerance, e.g. as GsulA (alcohol tolerant), GR51 (no hydrogen uptake), GR52 (GR51 derivative with wider range of electron donors such as lactate and pyruvate) and the like can be used to form the electricigenic biofilm. In one embodiment, the electrode can be functionalized to generate current in less than 24 hours. In one embodiment, the wastewater can be treated in a single chamber bioelectrochemical cell. In one embodiment the wastewater can be treated in a two-chambered bioelectrochemical cell.

In one embodiment, an electrode can be functionalized with the formation of an electricigenic biofilm. The electricigenic biofilm can be engineered to metabolize selected carbon sources. Pre-seeding of the electrode with selected electricigenic microbes can result in improving the coulombic efficiency of the electrode.

Various terms are defined herein. See also definitions in U.S. Pat. Nos. 9,716,287 and 10,074,867, both of which are incorporated herein by reference in their entireties. In case of a conflict in the meaning of various terms, the definitions provided herein prevail.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The terms “and/or” means one or all of the listed elements or a combination of any two or more of the listed elements.

The terms “comprises,” “comprising,” and variations thereof are to be construed as open ended—i.e., additional elements or steps are optional and may or may not be present.

The term “biomass” as used herein, refers in general to organic matter harvested or collected from a renewable biological resource as a source of energy. The renewable biological resource can include plant materials, animal materials, and/or materials produced biologically. The term “biomass” is not considered to include fossil fuels, which are not renewable.

The term “plant biomass” or “lignocellulosic biomass” as used herein, is intended to refer to virtually any plant-derived organic matter (woody or non-woody) available for energy. Plant biomass can include, but is not limited to, agricultural crop wastes and residues such as corn stover, wheat straw, rice straw, sugar cane bagasse and the like. Plant biomass further includes, but is not limited to, woody energy crops, wood wastes and residues such as trees, including fruit trees, such as fruit-bearing trees, (e.g., apple trees, orange trees, and the like), softwood forest thinnings, barky wastes, sawdust, paper and pulp industry waste streams, wood fiber, and the like. Additionally grass crops, such as various prairie grasses, including prairie cord grass, switchgrass, big bluestem, little bluestem, side oats grama, and the like, have potential to be produced large-scale as additional plant biomass sources. For urban areas, potential plant biomass feedstock includes yard waste (e.g., grass clippings, leaves, tree clippings, brush, etc.) and vegetable processing waste, such as glycerin-containing water.

The term “glycerin-containing water”, as used herein, refers to a liquid, such as water, containing any amount of glycerin (i.e., glycerol, a polyol). The liquid can contain other components, such as solids, alcohols, oils, salts and/or other components. Glycerin-containing water includes glycerin wastewater produced as a waste product of biodiesel fuel production or from ethanol biorefineries. Although glycerin wastewater can refer to either “crude glycerin wastewater” (i.e., “unrefined glycerin wastewater” which is glycerin wastewater in its initial state after separation from a biodiesel fuel product) or “refined glycerin wastewater” following treatment (typically in preparation for selling) which increases the concentration of glycerin to at least about 80% by volume, the processes described herein are useful with crude glycerin water, thus eliminating the need for refining glycerin wastewater in the conventional manner.

The term “wastewater” as used herein, refers to a liquid, such as water, containing any amount of components such as carbon waste products. Carbon waste products can include fermentative inhibitors such as organic acids, e.g. lactate, acetate, pyruvate and the like. The liquid can contain other components, such as solids, alcohols, oils, salts and/or other components. The wastewater includes waste product and requires remediation or removal of the waste product prior to reutilization of the water. Wastewater as referred to herein includes industrial waste water streams such as wastewater after evaporation of solids in a bioethanol refinery or glycerin-containing water generated in a biodiesel plant.

The term “biofuel” as used herein, refers to any renewable solid, liquid or gaseous fuel produced biologically, for example, those derived from biomass. Most biofuels are originally derived from biological processes such as the photosynthesis or fermentation processes and can therefore be considered a solar or chemical energy source. Other biofuels, such as natural polymers (e.g., chitin or certain sources of microbial cellulose), are not synthesized during photosynthesis or fermentations, but can nonetheless be considered a biofuel because they are biodegradable. Biofuels can be derived from biomass synthesized during photosynthesis. These include, for example, agricultural biofuels (defined below), such as biodiesel fuel. Biofuels can also be derived from other sources, such as algae, to produce algal biofuels (e.g., biodiesel fuel). Biofuels can also be derived from fermentations such as during the fermentation of sugars by yeasts (e.g., ethanol fuel). Biofuels can also be derived from municipal wastes (residential and light commercial garbage or refuse, with most of the recyclable materials such as glass and metal removed) and from forestry sources (e.g., trees, waste or byproduct streams from wood products, wood fiber, and pulp and paper industries). Biofuels produced from biomass not synthesized during photosynthesis also include, but are not limited to, those derived from chitin, which is a chemically modified form of cellulose known as an N-acetyl glucosamine polymer. Chitin is a significant component of the waste produced by the aquaculture industry because it comprises the shells of seafood.

The term “biodiesel fuel” or “biodiesel” as used herein, refers generally to long-chain (C₁₂-C₂₂) fatty acid alkyl esters, which are most often fatty acid methyl (FAMEs) or ethyl (FAEEs) esters. Biodiesel fuel can be produced from both agricultural and algal oil feedstocks. Biodiesel fuel is chemically analogous to petrochemical diesel, which fuels compression engines and can be mixed with petrodiesel to run conventional diesel engines. Petrodiesel is a fuel mixture of C₉ to C₂₃ hydrocarbons of average carbon length of 16, having approximately 75% of linear, branched, and cyclic alkanes and 25% aromatic hydrocarbons. In general, biodiesel and petrodiesel fuels have comparable energy content, freezing temperature, vapor pressure, and cetane rating. Biodiesel fuel also has higher lubricity and reduced emissions. The longer chain in FAEEs increases the cetane rating and energy content of the fuel, while decreasing its density, and pour and cloud points. As a result, combustion and flow properties (including cold flow properties) are improved, as is fuel efficiency. Once combusted, emissions and smoke densities are also minimized.

The term “agricultural biofuel”, as used herein, refers to a biofuel derived from agricultural crops (e.g., grains, such as corn), crop residues, grain processing facility wastes (e.g., wheat/oat hulls, corn/bean fines, out-of-specification materials, etc.), livestock production facility waste (e.g., manure, carcasses, etc.), livestock processing facility waste (e.g., undesirable parts, cleansing streams, contaminated materials, etc.), food processing facility waste (e.g., separated waste streams such as grease, fat, stems, shells, intermediate process residue, rinse/cleansing streams, etc.), value-added agricultural facility byproducts (e.g., distiller's wet grain (DWG) and syrup from ethanol production facilities, etc.), and the like. Examples of livestock industries include, but are not limited to, beef, pork, turkey, chicken, egg and dairy facilities. Examples of agricultural crops include, but are not limited to, any type of non-woody plant (e.g., cotton), grains such as corn, wheat, soybeans, sorghum, barley, oats, rye, and the like, herbs (e.g., peanuts), short rotation herbaceous crops such as switchgrass, alfalfa, and so forth.

The term “biodegradable”, as used herein, refers to a substrate capable of being decomposed, i.e., chemically broken down, by the action of one or more biological agents, such as bacteria.

The term “biocatalyst” as used herein, refers to organisms capable of metabolizing substrates.

The term “electricigen” or “exoelectrogen” as used herein, refers to a biocatalyst which is electrochemically active or an electricity-generating microorganism, i.e., an organism capable of transferring electrons to an electrode with or without mediators.

The term “bioprocessing microorganism” as used herein, refers to a microorganism capable of degrading biomass, such as lignocellulose substrates and glycerin-containing water.

The term “consolidated bioprocessing (CBP) organism” as used herein refers to a bioprocessing microorganism that is also capable of fermenting the degraded biomass into one or more biofuels, i.e., capable of performing a single step hydrolysis and fermentation. A CBP is useful for insoluble substrates that involve both a hydrolysis and fermentation step.

The term “fermentative organism” as used herein, refers to an organism capable of fermenting a substrate.

The term “alcohol-tolerant” as used herein, refers to a mutant of a microbial strain adaptively evolved or genetically engineered to have increased tolerance to alcohol as compared with the native microbe.

The term “glycerol-tolerant” as used herein, refers to a mutant of a microbial strain adaptively evolved or genetically engineered to have an increased tolerance to glycerol as compared with the native microbe.

The term “heat-tolerant” as used herein, refers to a mutant of a microbial strain adaptively evolved or genetically engineered to have an increased tolerance to heat as compared with the native microbe.

The term “adaptive evolution” as used herein, refers to the process that enhances the fitness of an organism to a particular environmental condition under appropriate selective pressure.

The term “ethanologenesis”, as used herein, refers to the metabolic process that results in the production of ethanol.

The term “fuel cell” as used herein, refers to a device used for the generation of electricity from a chemical and/or microbial reaction. The reaction can proceed naturally or can be facilitated with electrical input from, for example, a potentiostat. A fuel cell is comprised of anode and cathode electrodes connected through a conductive material. The electrodes may be housed in a single or double, i.e., separate chamber and when housed in a double, i.e., separate chamber may be separated by a cation- or proton-exchange membrane. A chemical or biological catalyst added to the anode drives electricity generation. A chemical or biological catalyst added to the cathode merges those electrons with protons in the medium to sustain the reaction at the anode.

The term “electrochemical cell” as used herein refers to a system in which an electrochemical reaction is occurring.

The term “bioelectrochemical system” or “BES” as used herein refers to an electrochemical system driven by microbes. A microbial fuel cell (MFC) and a microbial electrolysis cell (MEC) are each a type of bioelectrochemical system.

The term “bioelectrochemical cell” or “BEC” as used herein, refers to any type of electrochemical cell driven by biological catalysts, such as a microbial electrochemical cell, which is driven by microbial catalysts. BEC and BES will be used interchangeably in the description herein.

The term “microbial fuel cell” or “MFC” as used herein, refers to a fuel cell driven by electricigenic microorganisms either in a substantially pure (i.e., at least 90% purity) culture of at least a single species or in a mixed-species culture, i.e., a co-culture, which can include the electricigen at any concentration and a number of other species or as part of microbial consortia, i.e., a group of different species of microorganisms which may have different metabolic capabilities, but which act together as a community, such as a natural (e.g., biofilms) or defined laboratory microbial consortia. While the typical output of an MFC is electrical power, other bioproducts may also be produced. For example, a chemical or microbial reaction at the cathode can be used to synthesize chemicals or remove organic or inorganic contaminants.

The term “microbial electrolysis cell” or “MEC” as used herein, refers to a type of microbial electrochemical cell in which an electric current is input into a MEC to poise the anode electrode at a metabolic oxidizing potential that improves the performance of the electricigen and the removal of fermentative byproducts. As a result, the production of added-value fermentative products (e.g., hydrogen, methane, ethanol, PDO) from organic material (e.g., a polyol). In this way, electrical current produced at the anode is used to make hydrogen at the cathode. The same cocultures used in an MFC can be used in an MEC, often times with improved growth and catalytic activities.

The term “bioelectricity” as used herein, refers to electricity produced biologically, e.g., from biological materials such as biofuels and biomass.

The term “functionalization” as used herein refers to a modification of the surface chemistry of a material to enable new functions, features, capabilities and/or properties.

The term “functionalized electrode” as used herein refers to an electrode whose surface has been modified to enable new functions, features, capabilities and/or properties. Electrodes described herein, for example, are functionalized by the attachment of electricigenic bacteria on the surface of the electrode. The functionalization, for example, can couple the oxidative or reductive metabolism of the bacteria to the generation (anode) or consumption (cathode) of electricity at the electrode. It will be understood that “functionalized electrode” as used herein will be described in the context of a functionalized anode, which harvests electrons from the metabolic reactions catalyzed by the attached cells, but the functionalized electrode can also refer to a functionalized cathode that can be used in BESs. A functionalized anode electrode, for example, is when the electrode starts oxidizing the electron donors and generating current.

The term “biofilm” as used herein refers to one or more bacteria attached to the surface of the electrode. The bacteria can cover the surface of the electrode partially or completely and may be composed of a single, several or many layers of cells. Thus, the biofilm can vary in electrode surface coverage and thickness.

The term “electricigenic biofilm” as used herein refers to a biofilm formed by attachment of electricigenic bacteria to the surface of the electrode for transfer of electrons between the electricigenic bacteria on the electrode surface and the electrode. The electricigenic bacteria can be on the surface and/or cover the surface of the electrode partially or completely and may be composed of a single, several or many layers of cells. Thus, the electricigenic biofilm can vary in the amount of electrode surface it covers and the thickness of the biofilm formed on the surface. The electricigenic biofilm can include one or more types of electricigenic bacteria and may also include non-electricigenic bacteria. “Electricigenic biofilm” and “biofilm” will be used interchangeably in the description herein.

The term “condensate medium” or “CM” as used herein refers to a synthetic medium containing evaporate (process) condensate generated in a bioethanol refinery after centrifugation and drying of solids. Such wastewater contains acetate and lactate as well as other chemicals. One or more supplements (vitamins, minerals, salts, nitrogen source, and/or metals) for growth of the electricigenic microorganisms can also be added.

The term “growth media” as used herein refers to a mix of chemicals dissolved in a buffered solution that provide the necessary nutrients needed for a microbial cell to grow and divide when incubated at a viable temperature.

The term “DB medium” as used herein refers to a growth medium prepared by dissolving essential minerals, vitamins, and electron donors (such as acetate and lactate) in water. Electricity generated with a bioelectrode using a DB medium is known to be from the growth of the bioelectrode using the electron donors. A DB medium is designed for optimal growth of the electricigen Geobacter sulfurreducens (Gsu) and strains derived from it such as GsuA, GR51 and GR52, as described by Speers A. M. and G. Reguera, Appl Environ Microbiol, 2012, 78, 437-444, incorporated herein by reference in its entirety.

The term “functionalization” as used herein refers to a modification of the surface chemistry of a material to enable new functions, features, capabilities and/or properties.

The term “pre-seeding” as used herein refers to the process of adding electricigenic bacteria to the electrode environment in order to form an electricigenic biofilm of the selected bacteria on the electrode surface and the desired surface coverage and/or thickness. It is to be understood that the electrode surface is accessible to the cells during pre-seeding. It is to be understood that accessibility of the electrode surface refers to the availability of the electrode surface for the attachment of electricigenic bacteria.

The term “pre-seeding media” as used herein refers to a liquid or media that is used to functionalize an electrode in a chamber, e.g. CM.

The term “pre-seeding culture” as used herein refers to a culture of electricigenic bacterial cells that is cultured in growth media with selected substrates for use in an inoculum. This culture acclimates cells to the growth conditions.

The term “inoculum” as used herein refers to electricigen bacteria from the pre-seeding culture that has been harvested by, for example, centrifugation. The cells may be added directly to a chamber with an electrode in order to functionalize electrode. The cells may be resuspended in a compatible buffer prior to addition to a chamber for functionalization of an electrode.

The term “cell phase” as used herein refers to a phase in the bacterial cycle such as lag phase, exponential phase and/or stationary phase. The cells desirable for use as inoculum are in a cell phase that have cell surfaces suitable for attachment to the electrode by producing a component (e.g., exopolysaccharide with cytochromes) that improves their ability to stick to an electrode surface and start to grow on it by transferring respiratory electrons.

The term “exponential phase” as used herein refers to a period of cell growth characterized by cell doubling. The number of new bacteria appearing per unit time is proportional to the present. population. If growth is not limited, doubling will continue at a constant rate so both the number of cells and the rate of population increase doubles with each consecutive time period.

The term “stationary phase” as used herein refers to a period where the number of new cells created is limited and as a result the rate of cell growth matches the rate of cell death. Growth of cells may be due to growth-limiting factors such as the depletion of an essential nutrient, and/or the formation of an inhibitory product such as an organic acid.

The term “fastidious organisms” as used herein refers to microbes that metabolize one or more of the substrates present in the liquid with the electrode but are poorly electricigenic, not electricigenic at all, or compete for substrates that sustain the growth and activities of the desired electricigenic biofilm. Growth of fastidious organisms can divert substrates from electricigenic bacteria and decrease levels of current. Fastidious organisms may also attach to the electrode surface and prevent access of the electricigenic microorganisms to the electrode, also reducing the levels of current and, in some cases, substrate consumption by the electrode-attached biofilm. Decreasing the growth of fastidious organisms increases the availability of the substrates for metabolism by electricigenic bacteria and access to the electrode to use it as an electron acceptor and thus, increases the coulombic efficiency of the electrode.

The term “coulombic efficiency” as used herein refers to the amount of electron donor converted into electricity by the electrode-attached cells.

The term “substrates” as used herein refers to the molecules metabolized by the electricigenic bacteria that support the growth of the electricigenic cells on the electrode and that result in the transfer of metabolically generated electrons to the electrode.

The term “supplements” as used herein refers to molecules that are essential or enhance the growth of the electricigenic bacteria on the electrode and their ability to generate current. Supplements can include, for example, growth sustaining nutrients required such as vitamins, minerals, salts, nitrogen sources and the like. They can also include buffering agents and buffers needed to establish an optimal pH for growth and metabolic activity of the cells.

The term in “low chemical oxygen demand” or “low COD” wastewater as used herein refers to an aqueous solution with a concentration of organic carbon substrates such that the amount of oxygen necessary to fully oxidize the organic carbon pool to CO₂ and H₂O is low. In the description herein, solutions containing less than about 10,000 mg of COD per L are regarded as having low COD.

The term “high chemical oxygen demand” or “high COD” wastewater as used herein refers to an aqueous solution with a concentration of organic carbon substrates such that the amount of oxygen necessary to fully oxidize the organic carbon pool to CO₂ and H₂O is high. In the description herein, solutions containing about 10,000 or more mg of COD per L are regarded as having high COD.

Biodiesel can be produced from dedicated agricultural oil feedstocks, such as soybeans, with relatively low inputs and/or minimum impacts on existing agricultural practices, rural economies, and the environment. The economic and environmental viability of the biodiesel industry is, however, limited by the large volumes of glycerol-containing wastewaters generated during production, which most often need to be disposed of for a fee at water treatment facilities. Wastewater with approximately 40-50% of glycerol is generated after the phase separation of the crude biodiesel, but the glycerol is further diluted to ca. 10% after adding wastewater generated from the washing of the crude biodiesel. Glycerol prices have been traditionally high enough to allow producers to generate profit from refining the diluted glycerol waste, concentrating it to a ˜80% stock, and selling it to glycerol biorefineries. However, the rapid growth of the biodiesel industry in the last two decades has produced glycerol in excess of its demand and prices have dropped dramatically. In this saturated market, glycerol has become a very low-value or a waste product for biodiesel producers and glycerol-containing wastewaters are often an economic and environmental liability to the biodiesel industry. As such, unrefined glycerin wastewater is oftentimes disposed of as hazardous waste (e.g., containing hazardous concentrations of glycerol and methanol), which can be costly to the biodiesel producer.

In one embodiment, the ability of microorganisms to completely oxidize organic compounds to carbon dioxide (CO₂) with an electrode serving as the sole electron acceptor can be utilized to convert complex substrates, such as organic wastes and renewable biomass, to electricity and/or biofuels in bioelectrochemical systems (BESs) The degradation of complex substrates in BESs can mimic the natural process of organic matter degradation. In one embodiment, the degradation can involve the collective activities of microorganisms growing syntrophically to ferment and fully oxidize carbon substrates all the way to CO₂.

In one embodiment, during the treatment of wastewater, for example, natural microorganisms in the water stream can colonize the anode electrode of BES as a biofilm where fermentative and electroactive microorganisms can synergistically cooperate to degrade the organic substrates to CO₂ while generating electrical currents.

In some embodiments, enriching for biofilm-forming electricigens on the electrode can be advantageous for efficient wastewater treatment applications. In embodiments requiring continuous flow operation, electricigens that are attached as a biofilm may not bewashed away under flow. In contrast, unattached or loosely attached cells (not part of a biofilm) may be washed away in a continuous flow operation. These biofilm-forming electricigens may be cells already present in the wastewater and/or introduced externally to pre-seed the electrode prior to wastewater treatment. Promoting their attachment to and growth on the electrode over non-electricigenic or poorly electricigenic microorganisms may ensure that they remain active while oxidizing substrates and generating electricity under flow.

Treatment options exist in the art to enrich for microbial consortia on the anode electrode with microorganisms and nutrients present in the waste stream that is to be treated. These treatments require waste streams to have sufficiently high Chemical Oxygen Demand (COD) (e.g., >10,000 mg COD/L) to provide the nutrients and microbial loads needed to enrich for electrode-associated biofilms. Effective treatment of the wastewater also requires weeks (up to one month) to enrich for microbial consortia on the electrode with the activity needed to efficiently convert substantial amounts of substrates carried in the wastewater to CO₂ and electricity.

In some embodiments, the enrichment process can simultaneously select for fermentative and electricigenic partners as well as fastidious organisms that compete for electrode surface and nutrients. In one embodiment, enrichment in the biofilms of electricigens in the genus Geobacter can occur. These electricigenic bacteria can grow as an electroactive biofilm that efficiently couples the oxidation of fermentation products (e.g., acetate, lactate, formate, pyruvate and H₂) to electricity. BESs fed with fermentation products such as acetate often enrich for Geobacter electricigens.

In some embodiments, acetate can enrich for Geobacter over other bacteria. However, in some embodiments, formate and lactate, for example, may be present in the electrode environment during functionalization and this environment may not be effective for enriching for Geobacter over other bacteria. As a result, electrodes may be enriched for more fastidious (non-electricigenic) microbes that can reduce the coulombic efficiency of the electrode. In one embodiment, engineered and/or adaptively evolved strains of Geobacter that can efficiently oxidize these other fermentation products can be used. The use of engineered and/or adaptively evolved strains for pre-seeding can prevent the attachment of fastidious organisms and maximize the type and amounts of fermentation products that can be converted into electricity.

However, in prior art biofilms, growth of fastidious organisms often reduces the representation of Geobacter in the bioelectrodes and, as a result, BES performance. These fastidious organisms may compete for fermentation products such as lactate, which Geobacter cannot efficiently oxidize in BESs, acting as a sink of electrons and reducing BES performance. Fastidious organisms can also compete with Geobacter for the colonization of the electrode surface, reducing the availability of the electron acceptor that supports the growth of the electricigenic cells and the amount of current generated from their electricigenic activities.

In some embodiments, a method to pre-seed the anode electrode with Geobacter strains can result in development of robust bioelectrodes for BES applications. The strains of Geobacter used for pre-seeding can be strains genetically engineered and/or adapted for the efficient oxidization in a BES of common fermentation products such as acetate, lactate and pyruvate, which the natural strains in the genus cannot utilize efficiently.

In one embodiment, the functionalization of the BES anode electrodes with the electricigenic film can be achieved in less than 24 hours, or in less than 12 hours. This contrasts with the weeks-long enrichment processes that are often needed in the methods of the prior art to establish a mature bioelectrode for BES applications.

In one embodiment, the method described herein for functionalizing electrodes can maximize electrode surface coverage by the electroactive microorganisms, preventing fastidious organisms from colonizing the electrode. This can result in increasing the coulombic efficiency of the electrode.

In one embodiment, the functionality of the improved bioelectrodes can be used in BESs for the removal of fermentation inhibitors from waste streams recycled in an ethanol biorefinery plant. These waste streams can have too low a chemical oxygen demand (COD) and microbial loads to permit the natural enrichment of microbial consortia on BES anodes. Yet the functionalization of the anodes as described herein with electricigenic microorganisms can provide an efficient strategy for their treatment, thus expanding BES applications to the treatment of low COD wastes. In one embodiment, the rapid pre-seeding of a bioelectrode can permit the treatment of low COD wastewaters.

In embodiments for functionalizing electrodes with wastewater having low COD, the pre-seeding can provide the opportunity to treat wastewaters with COD that is too low to provide the nutrients needed to enrich for an active electricigenic biofilm on the electrode. Low COD waters can also have lower microbial loads to provide cells for the enrichment. But pre-seeding as described herein can bypass these limitations. In one embodiment of treating low COD wastewater in a BES, the electricigenic cells can be added first and then, if needed, the fermentative cells can be added to functionalize the electrode and permit the treatment of the low COD water.

The functionalization of the anode electrode using the methods described herein can pre-establish an electricigenic biofilm that can prevent fastidious organisms from accessing the electrode surface, an important consideration to improve the performance of BES treating high COD wastewaters. In embodiments for functionalizing electrodes in wastewater with high COD (>10,000 mg COD/L), pre-seeding the electrode as described herein can reduce the time needed to establish a natural electricigenic consortium on the electrode compared to the methods of the prior art wherein the wastewater has sufficiently high COD (>10,000 mg COD/L).

In one embodiment, the activities of the electricigenic cells in the electrode-associated biofilm (or bioelectrode) can be customized during pre-seeding and/or during growth of the inoculum to convert specific fermentation products into CO₂ and electricity. In one embodiment, the activities of the bioelectrode can be customized during pre-seeding and/or during growth of the inoculum to enrich for specific fermentative microorganisms during the treatment of wastewaters to maximize synergistic interactions with the bioelectrode and BES performance. In one embodiment, pre-seeding the electrode can reduce the time needed to establish a natural biofilm consortium for the electrochemical treatment of wastewaters, an important consideration for BES applications.

In one embodiment, BESs can include improved electrodes containing electricigenic biofilms to drive current generation efficiently while metabolizing substrates in wastes such as wastewater from bioethanol refineries. In one embodiment, the electrodes described herein can remove fermentation inhibitors from waste streams recycled in a bioethanol refinery.

In one embodiment, the present description includes a method of making electrodes. The method can include functionalizing electrodes for transfer of electrons between the electricigenic biofilm on the electrode surface and the electrode. The method can include functionalizing the electrodes for use in BESs. In one embodiment, the BESs can be driven by electrochemical activities of biofilms, such as anode biofilms. The BESs with the improved electrodes can be used, for example, for removal of organic acids derived from the fermentation of organic matter or other reactions. In one embodiment, the methods for making electrodes described herein can reduce the time needed to establish a functional bioelectrode, e.g. a biologically active anode (or bioanode). The method can also minimize electrode colonization by non-electroactive or poorly electroactive microorganisms. In one embodiment, the methods of making electrodes described herein can result in the rapid functionalization of electrodes with electricigenic microorganisms to reduce the time associated with bioelectrode development and maximize its electrochemical activity.

In one embodiment, the method can include providing an electrode with a suitable electrode material. In one embodiment, the electrode materials can be selected from any known conductive material, including, but not limited to, carbon, precious or non-precious metals, metal-organic compounds, stainless steel, conductive polymers, and the like, further including combinations thereof. In one embodiment, the cathode electrode material and the anode electrode material can be different materials. In one embodiment, each electrode can have any suitable configuration as is known to those skilled in the art, with each electrode having the same or a different configuration, as desired. In one embodiment, each electrode can have a configuration selected from one or more sheets (made from any conductive material), or one or more of various types of cloth, paper, glass, brush and rods, and the like, or any combination thereof.

The anode and cathode electrodes can be any suitable material. In one embodiment the anode electrode material is graphite. In some embodiments, the graphite can have a low specific resistance of 0.14 to 0.18 ohm/cm. In one embodiment, the graphite is Rayon felt graphite (Ceramaterial, N.Y.; 7×22 cm²). In one embodiment, the cathode can be carbon-based but any suitable carbon material can be used. In one embodiment, a highly conductive carbon-based cathode material with controllable pore size can be used. In one embodiment, the cathode material can be reticulated vitreous carbon. Any suitable reference electrode material can be used. In one embodiment, a silver-silver chloride reference electrode can be used. Other types of reference electrodes may be used and are within the scope of this description.

Any suitable material can be used for electrical connections. In one embodiment, materials can possess not only suitable conductive properties, but also resistance to corrosion and low toxicity (EC₅₀ values of more than 20000 mg/L for many microbes). In one embodiment, titanium wire can be used.

In one embodiment, the method may include placing the electrode in a chamber including a liquid. The chamber may include a substantial amount of liquid. In some embodiments, the chamber can be completely full, or at least 90 percent full, or 80 percent full, or 70 percent full, or less than 70 percent full. The electrode may be fully immersed, substantially immersed (about at least 70% or more immersed) or partially immersed (at least 10% immersed) in the liquid.

In one embodiment, the liquid in the electrode chamber can include substrates. The substrates can be molecules that are metabolized by the electricigenic bacteria of the biofilm. In one embodiment, the electricigenic bacteria can attach to the electrode surface to form the electricigenic biofilm, can oxidatively metabolize the substrates and can transfer electrons to the electrode. In one embodiment, the substrates can be organic acids. Organic acids can include lactate, acetate, formate, pyruvate and the like, provided alone or in combinations of two or more. In one embodiment, the substrates can be fermentative inhibitors and the oxidative metabolism of the fermentative inhibitors can allow fermentation to continue by removing the fermentative inhibitors from the liquid with concomitant generation of current.

In one embodiment, the method may include adding supplements to the liquid in the chamber that enhances the growth of the desired electricigenic bacteria. In one embodiment, the supplements can be added can include growth-promoting components or nutrients such as electron donors and acceptors, carbon and nitrogen sources, vitamins, salts, minerals, buffering agents and the like. In some embodiments, combinations of one or more of the supplemental components can be added.

In one embodiment, the liquid in the chamber can be water, e.g. wastewater that is to be treated. In one embodiment, the water samples or wastewater to be treated can be supplemented with a mix of minerals, salts and vitamins to provide optimal nutritional requirements for the growth of the electricigens on the anode electrode. The optimal nutritional requirements can vary depending on the selected electricigen bacteria and the contents of the components already present in the liquid. The supplements added to the liquid can vary and be determined, for example, by the electricigenic bacteria selected, growth conditions, and chemical contents and pH of the liquid.

In some embodiments, the supplements added may also be determined by the fastidious organisms that may be present in the electrode environment and may compete for nutrients with the electricigenic bacteria. In one embodiment, the supplements may be added to discourage the growth of fastidious organisms. In one embodiment, the supplements that encourage growth of fastidious organisms may not be added to the liquid.

In one embodiment, the supplements can include the mix of minerals, salts and vitamins that are similar to those in growth culture media such as in the DB media described, for example, by Speers et al. “Electron donors supporting growth and electroactivity of Geobacter sulfurrenducens anode biofilms.” Appl. Environ Microbiol. 78, 378-385, 2011, incorporated herein by reference in its entirety.

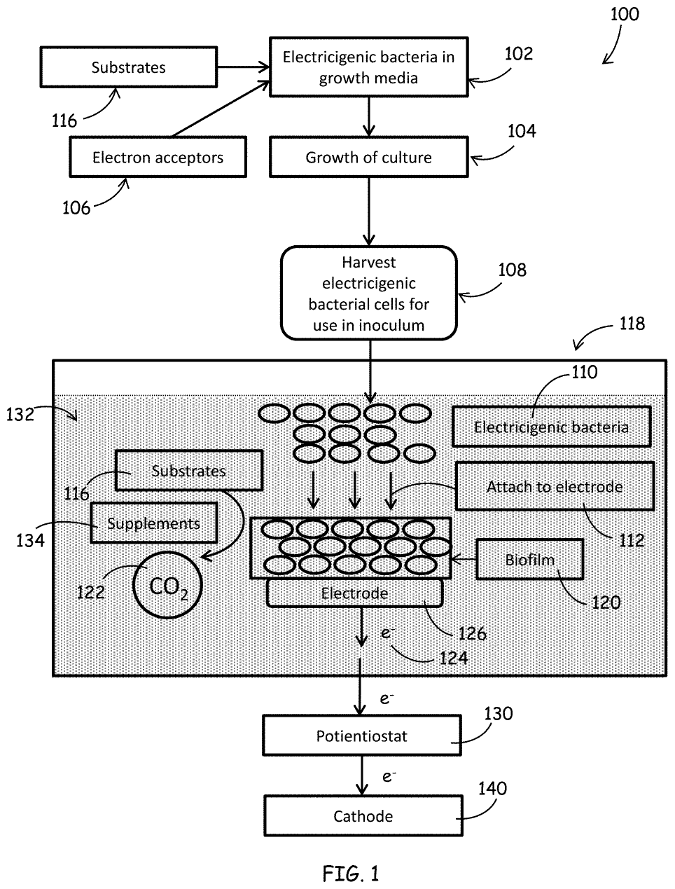

In one embodiment, a wastewater stream collected in an ethanol biorefinery was used instead of water and combined with minerals and vitamins to form a condensate media (CM). The wastewater stream collected from the ethanol biorefinery included acetate and lactate. Removal of acetate and lactate from the ethanol biorefinery process into the wastewater stream improves yeast fermentation and increases ethanol yields. The fermentation inhibitors present in the wastewater stream can be advantageously scrubbed, and used for production of electricity as described herein. As shown in FIG. 1, for example, use of the condensate medium (CM) generally aided formation of the bioelectrode and increased the current more than the standard laboratory DB medium supplemented with the same amount of acetate and lactate, although the amount of improvement varied. Without being bound by any theory, it is thought that the improved results with the condensate medium may be due to the additional chemicals in the waste stream.

In one embodiment, a nitrogen source may be added to the liquid. The nitrogen source can be, for example, NH₄Cl. In one embodiment, the NH₄Cl may be added at a concentration of 0.2 g/L. Other concentrations of NH₄Cl may be added and all are within the scope of this description. In one embodiment, a buffering agent may be added to the liquid in the chamber. In one embodiment, the buffering agent can be, for example, bicarbonate. In one embodiment, NH₄Cl and bicarbonate may be added to provide a nitrogen source and a buffering system in embodiments when CO2 is present in the reactor's headspace.

In one embodiment, the method for functionalizing an electrode can include poising the electrode at a potential that is optimal for the oxidative metabolism of the substrates in the liquid by the electricigenic bacteria of the electricigenic biofilm on the electrode. In one embodiment, the poising occurs by electrically connecting a potentiostat to the electrode and selecting a potential. In one embodiment, a positive potential of 240 mV versus a 3 M Ag/AgCl reference electrode is selected for oxidation of acetate and other fermentation products (e.g., lactate, formate, pyruvate and the like, alone or in combinations) by electricigenic bacteria such as Gsu and strains derived from it. The selection of the potential can vary and may depend on the type of electricigenic bacteria, the reference electrode selected and the substrates to be metabolized. As such, the potential selected can be determined based on the specific nature of the embodiment.

In one embodiment, the method can further include formation of the biofilm on the electrode surface by pre-seeding the electrode in the chamber with an inoculum of electricigenic bacteria. In some embodiments, the electrode in the chamber can be in a liquid and an inoculum of electricigenic bacteria can be added into the liquid of the chamber to encourage the formation of an electricigenic biofilm with the electricigenic bacteria of the inoculum.

In one embodiment, the inoculum can include one strain of an electricigenic bacteria. In one embodiment, the inoculum can include more than one strain of an electricigenic bacteria. In one embodiment, one inoculum with one type of electricigen bacteria can be added. In one embodiment, multiple inoculums can be added wherein each inoculum has a different strain(s) and/or different types of electricigenic bacteria.

The electricigenic strain for pre-seeding can be a biofilm-forming organism(s) that can grow and attach to the electrode surface as an electrochemically active biofilm to couple the oxidation of organic and inorganic substrates to the electricity. The electricigenic strain can be selected for its suitability to oxidize the range of substrates or electron donors that are the target for removal or metabolism in the liquid of the electrode chamber. The electricigenic bacteria can include, for example, Geobacter sulfurreducens (Gsu) or other electricigenic strains in the genus Geobacter and/or other biofilm-forming electricigenic genera.

In one embodiment, the electrode-associated electricigenic biofilm or bioelectrode can be an alcohol-tolerant strain of the electricigen G. sulfurreducens (GsulA) as described in Speers et al. “Fermentation of glycerol into ethanol in a microbial electrolysis cell driven by a customized consortium.” Environ Sci Technol 48, p.6350-6358. 2014, incorporated herein by reference. In one embodiment, the electricigenic strain can be G. sulfurreducens GR51, a GsulA derivative carrying a deletion in the gene encoding the uptake hydrogenase large subunit, hybL, to prevent the oxidation of H₂ generated at the cathode as described in Awate et al. “Stimulation of electrofermentation in single-chamber microbial electrolysis cells driven by genetically engineered anode biofilms.” J Power Sources 356, 510-518, 2017, incorporated herein by reference. In one embodiment, the strain can be G. sulfurreducens GR52, a GR51 derivative adaptively evolved for improved oxidation of lactate as described in Awate et al. “Stimulation of electrofermentation in single-chamber microbial electrolysis cells driven by genetically engineered anode biofilms.” J Power Sources 356, 510-518, 2017, incorporated herein by reference. The GR52 strain can advantageously also provide for improved oxidation of pyruvate compared to the GR51 strain. As such, GR52 strain can be used in embodiments for improved oxidation of pyruvate.

In one embodiment, the strain and/or strains grown for use in the inoculum can be selected based on their adaptability for the conditions in the chamber. In one embodiment, the liquid in the chamber can be wastewater that may include high amounts of fermentative inhibitor such as lactate. In such embodiments, the inoculum used for pre-seeding the electrode can include, for example, strain GR52 a strain adaptively evolved for improved oxidation of lactate. The adaptive evolution of improved lactate metabolism by GR52 can maintain the efficiency of acetate and formate metabolism native to Gsu and also permits the efficient use of pyruvate, another common fermentation product. In one embodiment, the inoculum for pre-seeding can only include GR52. In one embodiment, the inoculum for pre-seeding can include other strains such as GR51, GsulA and/or other strains in addition to GR52.

In one embodiment, the strain and/or strains grown for use in the inoculum can be selected and grown in the presence of selected substrates. In one embodiment, the substrates and conditions used in the growth of electricigenic bacteria in the inoculum can be similar to the substrates and conditions present in the liquid where the electricigenic biofilm is formed.

In one embodiment, the substrates present in the wastewater in the chamber can include fermentative inhibitors such as lactate and/or acetate. In such embodiments, electricigenic film rich in electricigenic bacteria that can oxidize acetate and/or lactate are desired. In one embodiment, the electricigenic bacteria for use as inoculum for pre-seeding the electrode can be grown in media supplemented with acetate and lactate. In one embodiment, the electricigenic bacteria in the inoculum and the conditions in which the electricigenic bacteria in the inoculum can be grown are based on the substrates present in the liquid of the chamber where the electricigenic biofilm is formed. In other words, the bacteria selected and the conditions for growing the inoculum parallel or partially parallel the conditions and/or substrates present in the liquid where the electricigenic biofilm is formed and/or is functional. Film formed under these conditions can contribute to increased coulombic efficiency.

In one embodiment, the method can include preventing or reducing the ability of fastidious organisms to grow on the electrode and/or compete for substrates. As described herein, fastidious organisms can grow in the presence of substrates that are not efficiently metabolized by electricigenic bacteria. Growth of fastidious organisms can remove substrates from the liquid without the concomitant generation of current since fastidious organisms are not electricigenic bacteria. In other words, the fastidious organisms can act as electron sinks. In one embodiment, the inoculum for pre-seeding can include electricigenic bacteria that can metabolize substrates that in prior electrodes may be metabolized by fastidious organisms. In one embodiment, reducing the population of fastidious organisms in the biofilm at the electrode can increase the coulombic efficiency of the functionalized electrode since a greater percentage of the biofilm can include electricigenic bacteria.

In one embodiment, a pre-seeding culture can include electricigenic bacteria which may be pre-grown in growth media for use in an inoculum in a separate vessel under conditions that can permit fast growth and cell acclimation to specific electron donors. The cells in the pre-seeding culture can then be harvested by, for example, centrifugation, washed in fresh medium, and resuspended in a liquid. In one embodiment, the liquid can be the same liquid used for pre-seeding the electrode. The cells may be resuspended at a volume of about 10% or more the volume of the pre-seeding liquid. In one embodiment, optimal pre-seeding culture growth can be achieved at 30 or 35° C. in an anaerobic mineral (DB) medium supplemented with acetate as electron donor, alone or in combination with other electron donors and carbon sources, and fumarate as electron acceptor, as described by Speers, A. M. and G. Reguera (2012) in “Electron donors supporting growth and electroactivity of Geobacter sulfurreducens anode biofilms.” Appl. Environ. Microbiol. 78(2): 437-444, incorporated herein by reference in its entirety.

The electricigenic bacteria in the pre-seeding culture may be in any cell phase when harvested to form the inoculum. The electricigenic bacteria in the pre-seeding culture may be in exponential phase or stationary phase when harvested to form the inoculum. The growth phase (exponential versus stationary phase) of the pre-seeding culture can be selected to harvest cells suitable for pre-seeding specific electrode materials. In one embodiment, stationary phase cells of Gsu and its variants can be used in the inoculum for pre-seeding carbon electrode materials.

The higher the OD, the greater number of cells available to colonize the electrode. In one embodiment, the stationary phase cells used as inoculum can be grown to an optical cell density or optical density (OD) at 600 nm from about 0.5 to about 0.7 prior to cell harvesting. In one embodiment, the stationary phase cells used as inoculum can be grown to an OD at 600 nm of about 0.6 prior to cell harvesting. In other embodiments, a lower OD can be used, such as less than 0.5 or less than 0.4 or no less than about 0.3. However, lower OD's require additional time for cells to attach to electrode and begin growing. An OD higher than 0.6 can also be used. In one embodiment, the cells can be washed once in fresh medium prior to resuspending them in the liquid where the electrode is immersed. In one embodiment, the optical density at 600 nm of the liquid containing the electrode and the inoculum can be between about 0.15 and about 0.3 for functionalizing the electrode in less than 24 hours. In one embodiment, the optical density at 600 nm of the liquid containing the electrode and the inoculum can be at least about 0.2 for functionalizing the electrode in less than 24 hours.

In one embodiment, electricigenic bacteria in the pre-seeding culture may be harvested in exponential phase. In one embodiment, the OD at 600 nm of the liquid containing the electrode and the inoculum can have an OD of at least about 0.2 for functionalizing the electrode in less than 24 hours.

In one embodiment, the pre-seeding culture cells can be grown in growth media containing the same electron donors as the electron donors present in the wastewater to be treated in order to acclimate the cells to utilize the electron donors. In one embodiment, the pre-seeding culture cells can be grown in DB medium with acetate and lactate as electron donors and fumarate as electron acceptor (DBALF) to harvest cells suitable for pre-seeding electrodes that can be used in BESs configured to treat evaporate (process) condensate from a bioethanol refinery rich in fermentative inhibitors such as acetate and lactate. In one embodiment, the pre-seeding culture cells can be grown in Condensate Medium (CM), a synthetic medium prepared by supplementing the evaporate (process) condensate with growth-promoting molecules and nutrients such as electron donors, carbon and nitrogen sources, vitamins, salts, minerals and buffering agents.

In one embodiment, the addition of a pre-seeding inoculum can lead to the formation of at least one layer of electricigenic cells in the biofilm on the surface and/or covering the surface of the electrode. In one embodiment, the addition of a pre-seeding inoculum can lead to the formation of multiple layers of electricigenic cells in the biofilm covering the surface of the electrode. In one embodiment, the pre-seeding inoculum can generate one or more layers of electricigenic bacteria that cover the surface of the electrode. This in turn can allow other bacteria to attach and form a thicker biofilm. These other bacteria can include electricigenic bacteria and/or other bioprocessing bacteria such as fermentative bacteria. Fastidious organisms may also grow but the growth of fastidious organisms may be minimized by providing competing electricigenic bacteria in the pre-seeding inoculum as described herein.

In some embodiments, the thickness of the electricigenic biofilm generated by pre-seeding with an inoculum may be specific of the wastewater to be treated. For embodiments for treating low COD wastewater, a thick electricigenic biofilm with electricigenic bacteria may be desired. In embodiments for treating high COD wastewater, an effective biofilm may grow on top of the one or more pre-seeded electricigenic layer, even if the electricigenic biofilm layer formed by pre-seeding is thin. Embodiments with high COD wastewater may allow for the pre-seeded electricigens to grow in thickness naturally and in consortia with other organisms.

In one embodiment, the bacterial consortia of the electrode-associated biofilm can include electricigenic bacteria that is greater than 10% of the bacterial consortia. In one embodiment, the electricigenic biofilm can include electricigenic bacteria that is greater than 25%, or greater than 50%, or greater than 75%, or greater than 85%, or greater than 90%, or greater than 95% of the bacterial consortia in the biofilm. In one embodiment, the bacterial consortia of the electrode-associated biofilm can include electricigenic bacteria that is between about 10% and about 95%, or between about 20% and about 95%, or between about 25% and about 95% of the bacterial consortia.

In one embodiment, the ratio of the electricigenic bacteria to fastidious organisms in the biofilm can be greater than about 1:1. In one embodiment, the ratio of the electricigenic bacteria to fastidious organisms in the biofilm can be greater than 2:1, or greater than 3:1, or greater than about 4:1, or greater than about 5:1, or greater than about 10:1.

In one embodiment, the method of making or functionalizing the electrode can be performed in a non-sterile environment. According to the state of the art, it is believed that if an electrode is to be functionalized with a particular bacteria that the biofilm formed on the electrode requires a sterile environment that only promotes the growth of the desired biofilm. In other words, it is believed that the contaminating microbes and/or fastidious organisms would compete for the substrates and a desired biofilm with high levels of electricigenic bacteria would form by the use of autoclaved media without the competition from fastidious organisms.

In contrast to the state of the art teachings, the method described herein can include the formation of the electricigenic biofilm for functionalizing electrodes using media, supplements, inoculum growth environment and conditions without the step of providing autoclaved media. The electrode can be functionalized by the formation of the electricigenic biofilm in a non-sterile environment such as without the use of autoclaved liquid and/or media.

In one embodiment, the pre-seeding culture growth medium can be autoclaved to sterilize it before growth of the inoculum. The inoculum can be added to a chamber with the electrode to be functionalized where the chamber includes non-autoclaved liquid media such as pre-seeding medium. In one embodiment, the pre-seeding medium in the chamber can be non-autoclaved CM media. The non-autoclaved pre-seeding growth medium can have nutrients for the electricigen but it is too restricted nutritionally to allow most other organisms to grow and to do so fast enough to outcompete the addition of electricigen in an inoculum. Pre-seeding of the electrode can also occur rapidly because strains that are acclimated to metabolize the nutrients rapidly are used.

In one embodiment, the method of making the electrodes can include functionalizing the electrodes. In one embodiment, the electrodes can be functionalized, e.g. start oxidizing the electron donors and generating current, in less than about one week. In one embodiment, the electrodes can be functionalized in less than about two days. In one embodiment, the electrodes can be functionalized in less than about 24 hours. In one embodiment, the electrodes can be functionalized in less than about 12 hours, or less than 8 hours, or less than 6 hours. In one embodiment, the electrodes can be functionalized in 5 hours or less, down to no more than about one hour. In various embodiments, the electrodes can be functionalized in a time range from about 1 to about 24 hours, such as about 5 to about 24 hours, such as about 6 to about 24 hours, such as about 8 to about 24 hours, such as about 5 to about 12 hours, such as about 6 to about 12 hours, such as about 6 to about 8 hours, including any ranges therebetween.

In one embodiment, the method can include forming an electricigenic biofilm of about 0.5 μm in thickness or greater. In one embodiment, the electricigenic biofilm formed is between about 0.5 and about 5 μm in thickness. In one embodiment, the electricigenic biofilm formed is between about 0.5 and about 50 μm in thickness. In one embodiment, the electricigenic biofilm formed is between about 0.5 and about 100 μm in thickness. In one embodiment, the electricigenic biofilm formed is between about 1 and about 50 μm in thickness. Electricigenic biofilms of greater than 100 μm up to about 100 μm are also within the scope of this description. It will be understood that in some embodiments the use of a porous electrode material such as porous graphite can lead to variability in the measurement of the thickness of the biofilms.

In one embodiment, the functionalized electrode can be used at the cathode to remove nitrate from wastewater and produce ammonia or nitrogen gas which exits the chamber. In other embodiments, the functionalized electrode is used as a cathode to reduce toxic metals and radionuclides. For example, a soluble uranyl cation can be reduced to a U(IV) mineral that can be recovered for disposal.

In one embodiment, the method can include forming a functionalized electrode that can generate current density. In one embodiment, the method can functionalize electrodes that generate current density of at least about 50 μA of current per cm² of electrode surface. In one embodiment, the electrode can generate at least about 100 μA of current per cm² of electrode surface. In one embodiment, the electrode can generate at least about 150 μA of current per cm² of electrode surface. In one embodiment, the electrode can generate at least about 250 μA of current per cm² of electrode surface. In one embodiment, the electrode can generate at least about 500 μA of current per cm² of electrode surface. In one embodiment, the current density can be generated in less than 24 hours, or less than 12 hours, or less than 8 hours, or less than 6 hours. In one embodiment, the current densities herein can be generated in about 5 hours or less, down to no more than about one hour. In various embodiments, the current densities can be generated in a time range from about 1 to about 24 hours, such as about 5 to about 24 hours, such as about 6 to about 24 hours, such as about 8 to about 24 hours, such as about 5 to about 12 hours, such as about 6 to about 12 hours, such as about 6 to about 8 hours, including any ranges therebetween.

In one embodiment, the method can include forming a functionalized electrode that can generate at least about 50 μA of current per cm² of electrode surface in less than about 8 hours. In one embodiment, the electrode can generate at least about 100 μA of current per cm² of electrode surface in less than about 8 hours. In one embodiment, the electrode can generate at least about 250 μA of current per cm² of electrode surface in less than about 8 hours. In one embodiment, the electrode can generate at least about 500 μA of current per cm² of electrode surface in less than about 8 hours.

It will be understood that the current and/or current density is limited by the availability of the substrates in the liquid of the electrode chamber. Depletion of the substrates in the chamber from the electrode environment can deplete the generation of the current and/or current density. In one embodiment, current may be generated for a few hours until most of the electron donors are consumed and then the current can begin to decrease. Addition of a new source of substrate can restart the generation of current by the electrode in a BES.

In one embodiment, wastewater can be treated. In one embodiment, the method can include generating an electrode with an electricigenic biofilm comprising electricigenic bacteria. In one embodiment, the electricigenic bacteria can have traits such as the ability to utilize the substrates present in the wastewater. In one embodiment, an electrode with the electricigenic biofilm can be first generated in a chamber with liquid. The liquid can be media such as DB media, CM media as described herein. Other media may also be used and are within the scope of this description. Supplements may be added to the liquid to be treated to enhance the growth of the electricigenic bacteria. The bioelectrode generated can then be incorporated into a BES for treatment of wastewater.

In one embodiment, the electrode can be functionalized by placing the electrode in the wastewater to be treated. Desired substrates and/or supplements can be added to the wastewater. The inoculum for pre-seeding can be added to the wastewater supplemented with the desired substrates and/or supplements to form an electricigenic biofilm to functionalize the electrode. The functionalized electrode can be used to treat the wastewater. In one embodiment, the electrode may be used to treat wastewater in a BES with a batch format.

In one embodiment, the electrode can be functionalized in wastewater and the wastewater can be treated in a BES with a continuous flow process. In this embodiment, the wastewater can be introduced into the BES through an inlet and allowed to be in contact with the electrode. In one embodiment, inoculum of desired electricigenic bacteria and/or supplements may be added to the wastewater in the BES. The microbes in the wastewater can attach to the electrode surface and begin to grow. The flow of wastewater from inlet to outlet until a biofilm is established on the electrode that removes the COD and generates electricity. The cells used to functionalize the electrode can attach in a matter of hours and stick to the electrode, so they don't wash off when the flow of wastewater is started. In one embodiment, the electrode can be functionalized in the wastewater in less than 24 hours. In one embodiment, the electrode can be functionalized in the wastewater in less than 12 hours.

In one embodiment, the functionalized electrode can be used for treatment of low COD wastewater. In these embodiments, the electrode can be functionalized by pre-seeding in media, such as condenstate medium (CM) media, containing selected substrates and/or supplements to enhance the growth of the electricigenic bacteria with the traits of interest such as metabolism of acetate and lactate. The functionalized electrode can then be introduced to the wastewater to be treated.

In one embodiment, the functionalized electrode can be used for treatment of high COD water. Pre-seeding the electrode can precede other pretreatment approaches currently used for the enrichment of microbial consortia on the electrodes in BESs to minimize the attachment and growth of fastidious organisms on the electrode. In one embodiment, pre-seeding the electrode can reduce the time needed to establish electricigenic consortia from the wastewater and improves substrate conversion rates and current generation.

In one embodiment, FIG. 1 shows process 100 for functionalizing an electrode to make the improved electrodes described herein. In the embodiment shown in FIG. 1, a process 100 is provided in which anode chamber 118 is pre-seeded with inoculum 108 to form biofilm 120 on electrode 126.

In one embodiment, as shown in step 102, electricigenic bacteria 110 are placed in growth media 102 and allowed to grow to a desired cell density for example, for about 24, 36, or 48h. In one embodiment, growth media 102 may be supplemented with substrates 116. In one embodiment, substrates 116 are the same substrates 116 present in liquid 132. In one embodiment, the bacteria 110 from growth culture 104 are harvested from culture 104 by, for example, centrifugation and used as inoculum 108. In one embodiment, inoculum 108 includes centrifuged cells of bacteria 110 that are added to liquid 132 in chamber 118. In one embodiment, inoculum 108 may include centrifuged cells of bacteria 110 resuspended in a small volume of media compatible with bacteria 110 and/or liquid 132 prior to addition to liquid 132.

When added to liquid 132, electricigenic bacteria 110 in inoculum 108 grow and form biofilm 120 attached to electrode 126. By attachment, it is meant that the biofilm is associated with the electrode and stays associated with the electrode surface even, for example, during a continuous flow operation. In one embodiment, attachment of the electricigen is by secreted molecule such as exopolysaccharide or proteins of the cells outer surface such as cytochromes. In one embodiment, attachment of the electricigen is via conductive protein filaments termed “pilus nanowires” that allow substantial stacking of cells on the electrode and efficient electron flow across the electricigenic film and to the electrode. Bacteria that generate protein filaments includes, but is not limited to, members of the Geobacteraceae family, such as G. sulfurreducens.

Anode chamber 118 includes liquid 132. Liquid 132 can be water, media, wastewater or other liquid that enables the growth and/or metabolic activity of electricigenic bacteria 110. Liquid 132 can include substrates 116 that are oxidatively metabolized by bacteria 110 to generate current. Substrates 116 can be for example, organic acids such as lactate and acetate. Liquid 132 can include other substrates 116 that are metabolized by electricigenic bacteria 110 and all are within the scope of this description. Additional substrates 116 and/or supplements 134 may be added to liquid 132 to optimize the growth of electricigenic bacteria 110. Electricigenic bacteria 110 attach to the electrode 126 and metabolize substrates 116 to CO₂ using electrode 126 as electron acceptor. The metabolism of the substrates 116 allows the bacterial cells 110 to grow as a biofilm 120 on the electrode 126 while discharging respiratory electrons 124 to electrode 126.

Electrode 126 housed in the anode chamber 118 is connected to potentiostat 130 to poise electrode 126 at a potential that is amenable to oxidative metabolism by electricigenic bacteria 110 in biofilm 120. In various embodiments, the electrode is amenable to oxidative metabolism in the presence of electron donors. Optimum potential is considered to be a potential which leads to maximum current during the oxidation of a specific electron donor or electron donor mix at a particular temperature and in a particular growth medium. In various embodiments, the electrode is poised at a level which is more positive than the redox potential of the half-cell reaction of the electron donor. In one embodiment, the substrate is acetate and the potential is greater than −290 mV (versus standard hydrogen electrode). In one embodiment wherein Gsu is the electricigen and acetate is the electron donor, the positive potential ranges from about 445 to about 505 mV (versus standard hydrogen electrode). Potentiostat 130 is also connected to cathode 140. The connection of the anode electrode 126 and cathode electrode 140 allows electrons 124 to reach cathode 140.

The functionalized anode electrode can be housed in any type of BES. In one embodiment, the anode and cathode electrodes are housed in separate chambers. In one embodiment, the anode and cathode electrodes are separated by a cation- or proton-exchange membrane. Spacing between the anode electrode and the cathode electrode can also vary, as is understood by those skilled in the art.

In one embodiment, the electrode functionalized by the methods described herein can serve as the anode in a BES. In one embodiment, the electrode functionalized by the methods described herein can serve as the cathode in a BES.

In one embodiment, the anode and cathode electrodes of the electrochemical cell are housed in the same chamber. In one embodiment, the BES can be a single chamber microbial electrolysis cell (SCMEC), that is, a BES reactor equipped with an anode, cathode and reference electrode connected to a potentiostat to poise the anode at a potential optimal for the growth of the electricigenic organisms. See, for example, Microbial biofilm voltammetry: direct electrochemical characterization of catalytic electrode-attached biofilms. Marsili E, Rollefson J B, Baron D B, Hozalski R M, Bond D R. Appl Environ Microbiol. 2008 December: 74(23):7329-37. Epub 2008 Oct. 10, which is hereby incorporated by reference in its entirety.

In one embodiment, an external or air-breathing cathode electrode is used. In this embodiment, the cathode chamber is removed and the cathode electrode is placed externally and in direct contact with the proton-exchange membrane. See, for example, Improved fuel cell and electrode designs for producing electricity from microbial degradation, Park D H, Zeikus J G. Biotechnol Bioeng. 2003 Feb. 5:81(3):348-55 and Electrically enhanced ethanol fermentation by Clostridium thermocellum and Saccharomyces cerevisiae. Shin H S, Zeikus J G, Jain M K., Appl Microbiol Biotechnol. 2002 March: 58(4):476-81, both of which are incorporated herein by reference in their entireties.

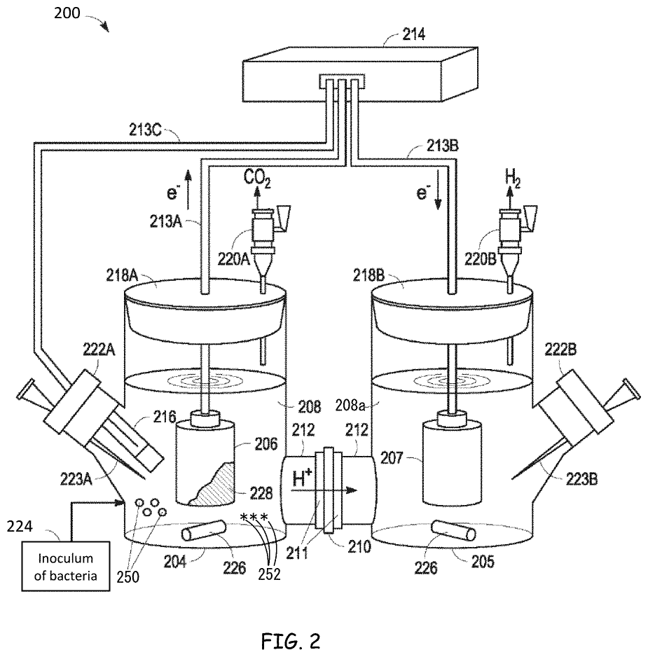

In the embodiment shown in FIG. 2, an H-type microbial electrochemical cell 200 is provided, which comprises two chambers (i.e., anode chamber 204 and cathode chamber 205) in an “H” configuration. The anode electrode 206 is located in the anode chamber 204 and the cathode electrode 207 is located in the cathode chamber 205. Anode electrode 206 is an electrode functionalized by pre-seeding with electricigenic bacteria 224 as described herein. A cation- or proton-exchange membrane 210, together with gaskets 211 and glass flanges 212, create a “glass bridge” which separates the anode and cathode chambers, 204 and 205, respectively.

In this embodiment, each chamber, 204 and 205 contains an amount of growth medium 208 and 208a, respectively. In one embodiment, Growth media 208 and 208a are substantially identical. The growth medium 208 can be any medium that supports growth of the electricigenic bacteria 224. Growth media 208 and 208a do not necessarily need to be the same in each chamber, 204 and 205.

In one embodiment, the growth medium 208 is fresh water (FW) (See G. Reguera, K. P. Nevin, J. S. Nicoll, S. F. Covalla, T. L. Woodard and D. R. Lovley. 2006. “Biofilm and nanowire production lead to increased current in microbial fuel cells”. Appl Environ Microbiol. 72(11): 7345-7348), which is incorporated herein by reference in its entirety. In one embodiment, growth medium 208 is condensate medium (CM) as described herein. In one embodiment, “Regan' s medium” is used as the growth medium 208. (See Ren, Z., T. E. Ward, and J. M. Regan. 2007. Electricity production from cellulose in a microbial fuel cell using a defined binary culture. Environ. Sci. Technol. 41:4781-6, hereinafter “Regan”) which is incorporated herein by reference in its entirety. In one embodiment, “Daniel Bond's medium” is used as the growth medium 208. (See Speers, A. M. and G. Reguera (2012). “Electron donors supporting growth and electroactivity of Geobacter sulfurreducens anode biofilms.” Appl. Environ. Microbiol. 78(2): 437-444.), which is hereby incorporated herein by reference in its entirety. In one embodiment, the growth medium 208 and 208a are present in the anode chamber 204 and cathode chamber 205 in substantial quantities so all the electrodes are fully immersed.

The anode electrode 206 and the cathode electrode 207 are electronically connected via anode conductive wires and cathode conductive wires, 213A and 213B, respectively, both of which, in turn, are connected to a potentiostat 214. The anode chamber 204 further houses a reference electrode 216, which is also connected to the potentiostat 214 with conductive wires 213C, as shown in FIG. 2.

The anode chamber 204 and cathode chamber 205 are sealed with an anode stopper 218A and a cathode stopper 218B, respectively. An anode outlet port 220A is provided in the anode stopper 218A and a cathode outlet port 220B is provided in the cathode stopper 220B. The anode chamber 204 is further equipped with an anode sparging port 222A into which a first needle 223A can be inserted. Similarly, the cathode chamber 205 is equipped with a cathode sparging port 222B into which a second needle 223B can be inserted. The sparging ports, 222A and 222B, further include suitably sized stoppers, as is known in the art.

In use, the potentiostat 214 poises the anode electrode 206 at a defined potential, thus allowing for a cathode-unlimited system for controlled and reproducible results. In one embodiment, the process begins by adding an inoculum of electricigenic bacteria 224 to the anode chamber 204 to initiate the generation of current.

In one embodiment, bacteria inoculum 224 can include one or more biocatalysts. Biocatalysts can include, for example, fermentative bacteria, electricigenic bacteria, glycerol metabolizing bacteria and the like. In one embodiment, inoculum 224 can include fermentative bacteria in addition to the electricigenic bacteria. The fermentative and electricigenic bacteria can be added at substantially the same time or sequentially. In an embodiment, with a single step hydrolysis and fermentation of the biomass 252 may be performed.

In one embodiment, the anode outlet port 220A also allows carbon dioxide (CO₂) to be vented out of the anode chamber 204 during the fermentation portion of the single step hydrolysis and fermentation. In one embodiment, the CO₂ is collected and recycled for use in an off-site process.

Fermentation byproducts comprising primarily one or more organic acids (substrates 250) and an amount of hydrogen gas (H₂) produced with the single hydrolysis and fermentation step are exposed to electricigenic biofilm 228 grown on anode electrode 206. In one embodiment, electricigenic biofilm 228 is generated prior to incorporation into BES 200. In one embodiment, electricigenic film 228 is formed in the chamber due to the pre-seeding of electrode 206 with inoculum 224 of electricigenic bacteria. Multiple inoculums may be added, for example, an inoculum of electricigenic bacterial may be added first and electricigenic film 228 allowed to form prior to the addition of an inoculum of fermentative bacteria. Electricigenic film 228 can grow to any suitable thickness. In one embodiment, electricigenic film 228 is at least about 40 to about 50 micrometers thick.

The electricigenic biofilm 228 catalyzes the split of electrons (e⁻) and protons (H⁺) present in the fermentation byproducts. The electrons (e⁻) then flow from the anode electrode 206 towards the cathode electrode 207 (such as through conductive wires 213A, into the potentiostat 214, and into conductive wires 213B, as shown in FIG. 2) whereas the protons (H⁺) permeate the proton-exchange membrane 210. With an adequate electrical input, the potential between the anode and cathode electrodes can be set so protons (H⁺) react with the electrons (e⁻) at the cathode electrode 207, thereby generating hydrogen gas (H₂). In the embodiment shown in FIG. 2, the hydrogen gas (H₂) generated in the cathode chamber 205 exits through the outlet port 220B.

Both sparging ports, 222A and 222B, are configured to remove oxygen gas, facilitate mixing, and/or provide defined gases for buffering the pH of the growth medium (e.g., CO₂-containing gas to buffer the pH of bicarbonate-containing medium) from their respective chambers, 204 and 205, and, ultimately from the microbial electrochemical cell 118. Mixing also can be achieved with stir bars 226 and other rotating devices, as is known in the art.

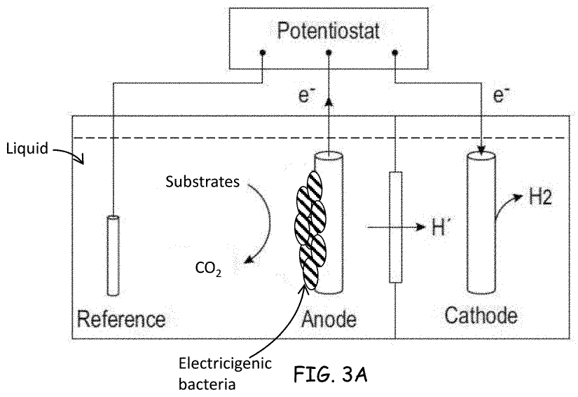

Simplified schematic of embodiments of MECs are shown in FIG. 3A-3C. The MECs comprise the same components as discussed above (shown in FIG. 2) except that, in various embodiments, anaerobic conditions are maintained at all times, and the potentiostat is connected to cathode and anode electrodes to provide external voltage.

In one embodiment, a single chamber MEC capable of operating under anaerobic conditions is used. (See FIG. 3C). In one embodiment, the single chamber MEC (SCMEC) can be scaled-up for commercial use with low cost materials and simplified design as compared to two-chambered MEC. In one embodiment, the SCMEC uses a single glass chamber of various volumes (e.g., 200 mL-2 L). In contrast to a conventional H-type MEC, the SCMEC does not rely on a proton exchange membrane to keep cathode and anode separate. SCMEC can include a potentiostat electrically connected to the anode, the cathode and the reference electrode. In one embodiment, anode can be incorporated into the SCMEC after the formation of an electricigenic biofilm. In one embodiment, the electricigenic biofilm is generated in the SCMEC by the methods described herein. The method includes, for example, poising the potential at the anode, addition of liquid to the chamber, pre-seeding with an inoculum of electricigenic bacteria and addition of supplements. The SCMEC advantageously can have reduced costs associated with operation. It can also be scaled up to larger volumes.

In embodiments shown in FIG. 3A and 3B, the anode and cathode electrodes are housed in separate chambers. In one embodiment, the anode and cathode electrodes are separated by a cation- or proton-exchange membrane. Spacing between the anode electrode and the cathode electrode can also vary, as is understood by those skilled in the art.

In one embodiment shown in FIG. 3A, substrate containing liquid such as wastewater is used as feedstock to generate electricity in a microbial electrochemical cell. In this embodiment, therefore, the substrate can be, for example, acetate, lactate and the like. The substrates are metabolized by the electricigenic bacteria in the biofilm covering the anode. Metabolism of the substrates by the electricigenic bacteria results in transfer of electrons to the anode and concomitant generation of current.

In one embodiment shown in FIG. 3B, glycerol-containing water is used as feedstock to generate ethanol and/or electricity in a microbial electrochemical cell. The glycerin-containing water can be subjected to one or more pretreatment steps to remove unwanted components, such as oils and salts (while retaining both glycerol and, if present, alcohol). In one embodiment, the pretreatment additionally or alternatively includes a concentration step to increase concentration of the glycerol in the glycerin-containing wastewater to a desired level.

In this embodiment, therefore, the substrate is glycerol present in glycerin waste. As such the glycerin can be provided to a BES, such as a MFC and a MEC. The glycerin is then degraded, i.e., metabolized and/or fermented in a single-step process using a glycerol-consuming microbe to produce an alcohol (e.g., ethanol, 1,3-propanediol) and fermentation byproducts such as hydrogen gas (H₂) and organic acids. In one embodiment as shown in FIG. 3B, the consortium at the anode electrode is a biofilm that includes electricigen G. sulfurreducens strains or other organisms capable of converting substrates such as organic acids (e.g. acetate, lactate, formate) to CO₂ with the concomitant generation of electricity.

The hydrogen gas and/or organic acids provide a source of electrons to the electricigenic bacteria in the electricigenic biofilm as described herein. Glycerin-containing biomass embodiments provide for use of a fermentative organism which cannot only produce fermentation products for the electricigenic cells , but can also serve as a biocatalyst to produce organic acids and/or alcohols that the electricigen cannot use. In one embodiment, the glycerin-fermenting organism serves as an ethanologenic biocatalyst. In one other embodiment, the glycerin-fermenting organism serves as a biocatalyst for the production of 1,3-propanediol (PDO).

In one embodiment, the alcohologenic biocatalyst is Clostridium cellobioparum (Cce) which can ferment glycerol into alcohols (such as ethanol and/or PDO) and fermentation byproducts that can be converted into electricity with Gsu as the electricigen. In one embodiment, a glycerol-tolerant strain of Cce (CceA) or an alcohol-tolerant strain of Gsu (GsuA) or a co-culture of Cce-Gsu, CceA-GsuA or any combination thereof, including any combination with Cce, is used as the alcohologenic biocatalyst.

In one embodiment, the processes described above are scalable up 10, 100 to 1000 times or more for large-scale ethanol and electricity production. In one embodiment, the electricity generated can be used to replace some of the electricity demand of a biofuel production facility, such as an ethanol and/or biodiesel production facility. In one embodiment, electricity is produced using a microbial electrochemical cell. In one embodiment, the ethanol produced according to the methods described herein can be distilled in a biodiesel production facility using existing distillation equipment and reused as the alcohol in the transesterification reaction.

Embodiments described herein include computer-implemented systems and methods operating according to particular functions or algorithms which may be implemented in software or a combination of software and human implemented procedures. In one embodiment, the software may comprise computer executable instructions stored on computer readable media such as memory or other type of storage devices. Further, such functions correspond to modules, which are software, hardware, firmware or any combination thereof. Multiple functions may be performed in one or more modules as desired, and the embodiments described are merely examples. The software may be executed on a digital signal processor, ASIC, microprocessor, or other type of processor operating on a computer, i.e., a computer system, such as a personal computer, server or other computer system.

In other embodiments, the in situ generation of bioproducts in a microbial electrolysis cell (MEC), such as ethanol and PDO, from polyol-containing biomass, such as glycerin-containing water, such as glycerin wastewater (e.g., crude or partially refined glycerin wastewater from a biodiesel production facility), is provided. It is to be understood that the various materials (e.g., consortia) and processes and considerations described above may, in various embodiments, be useful in this embodiment.

In one embodiment, this generation is driven by the synergistic metabolisms of a first biocatalyst, namely a fermentative microbe (i.e., bacterium) (e.g., Clostridium cellobioparum) and a second biocatalyst, such as an electricigen (e.g., Geobacter sulfurreducens). In one embodiment, the MEC can ferment glycerol into ethanol at high yields (e.g., 90% or greater) and produce fermentative byproducts that serve as electron donors for the electricigen.

In the embodiments, described herein, MECs driven by customized consortia are provided. In various embodiments, the MECs as shown in FIG. 3B can ferment glycerol when provided at a suitable loading, such as about 5 to about 15 wt %, such as about 8 to about 12 wt %, including any range or value therebetween, such as no less than or no more than 10 wt %,. In one embodiment, the glycerol loading is comparable to the loading in glycerin wastewater streams.

Any suitable consortium can be used. In one embodiment, the consortium includes Clostridium cellobioparum, a glycerol-fermenting bacterium selected for its superior ethanologenesis from glycerol, and the electricigen G. sulfurreducens, which can convert waste byproducts of glycerol fermentation into electricity. Optimization of the glycerol tolerance of the microbial catalysts via adaptive evolution and of the growth medium can, in one embodiment, result in a robust MEC platform that further stimulates glycerol consumption and ethanol production.

Any suitable materials can be used for the cathode, anode and reference electrode. In one embodiment, further cost reduction can be achieved by using cheaper cathode and anode materials having a surface area to volume ratio that is at least a magnitude of order higher (such as about 10 to 15 times higher) as compared with a conventional cathode and anode (e.g., surface area of around 150 cm² per cubic of graphite felt compared to surface area of around 6 cm² per cubic volume of graphite rods). The higher surface area to volume ratio of such electrodes provides for a cost-effective scale up of the system in terms of using an increased amount of second biocatalyst or electricigen bacteria (e.g., such as about 3 to about 10, such as about 4 to 8 or such as about 4 to 6 times, including any range therebetween, further including at least 5× less) and generating an increased amount of electricity (e.g., such as about 15 to about 35 times, such as about 20 to 35 times, or such as about 28 to 32 times, further including at least 30× more) while only providing a modest increase in size of electrodes (such as about 3 to about 9 times, about 4 to about 8 times, about 5 to about 7 time, further including at least 6× greater).

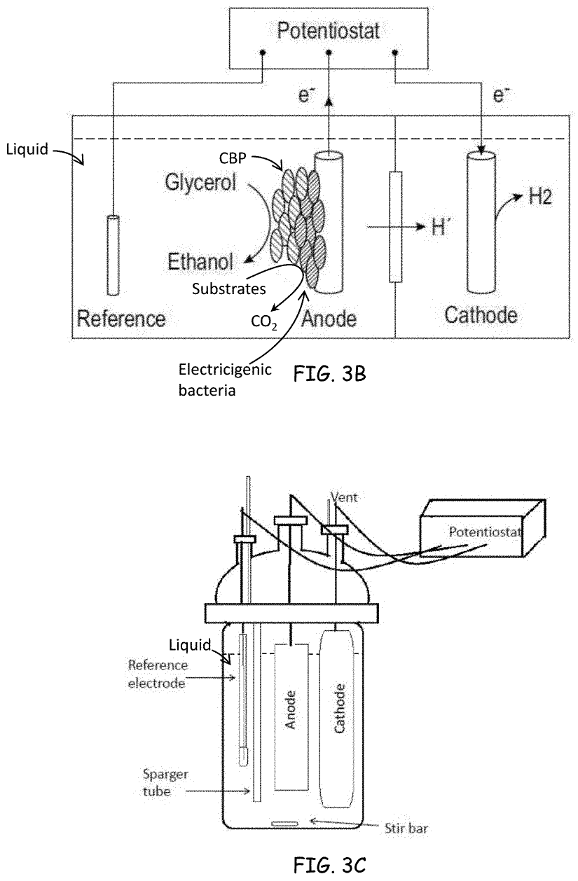

FIG. 4 shows one embodiment of a process for treating biomass and generating electricity in BESs equipped with the improved electrodes described herein. In the embodiment shown in FIG. 4, a process 400 is provided in which lignocellulosic-containing biomass 402 is subjected to one or more pretreatment steps to separate lignin 406 from insoluble cellulose/hemicellulose (hereinafter “insolubles”) 408.

In the embodiment shown in FIG. 4, the insolubles 408 are provided to a microbial electrolysis cell (MEC) 418 where they are degraded, i.e., hydrolyzed and fermented in a single-step process using a consolidated bioprocessing (CBP) microbe 410 to produce ethanol 412 and fermentation byproducts such as hydrogen gas (H₂) 414 and organic acids 416 and PDO. The hydrogen gas 414 and/or organic acids 416 provide a source of electrons 424 to support the growth of an electricigen 420, which gains energy by transferring electrons 424 to an electrode 426, thereby producing electricity 424 and a carbon dioxide (CO₂)-containing waste stream 422. Electrode 426 is formed by pre-seeding the electrode with electricigen 420 to form an electricigen biofilm as described in the methods disclosed herein.

Unlike conventional cellulosic ethanol processes which require separate hydrolysis and fermentation steps, embodiments described herein provide for use of a CBP organism 410 which is not only capable of catalyzing the enzymatic hydrolysis, but can also serve as an alcohologenic biocatalyst (alcohologenesis). In one embodiment, the CBP organism 410 serves as an alcohologenic biocatalyst (e.g., an ethanologenic biocatalyst). As such, the embodiments described herein are not reliant on a previous biomass solubilization step or previous growth of the CBP organism 410 and electricigen 420 in separate vessels prior to initiation of the fermentation process. Furthermore, the use of ethanologenic microorganisms in conventional methods that produce fermentation byproducts other than those that the electricigen can convert into electricity results in reduced electricity production and feedback inhibition of the fermentation by the CBP organism 410. In embodiments described herein, both the CBP organism 410 and the electricigen 420 can be simultaneously inoculated or sequentially inoculated in the same reactor while maintaining the net production of ethanol and electricity.

Any suitable biomass, as defined herein, can be used. In one embodiment, the biomass is a non-food biomass, such as agricultural waste. In one embodiment, corn stover is used. Additional steps known in the art may also be used to prepare the biomass for use in the novel process, including, but not limited to, milling. Process 400 is described in U.S. Pat. No. 10,074,867 to Reguera et al., incorporated herein by reference.

In one embodiment, syntrophic cooperation stimulates glycerol consumption, ethanol production, and conversion of fermentation byproducts into cathodic H₂ in a MEC. In one embodiment, the platform is improved by adaptively evolving glycerol-tolerant strains with robust growth at glycerol loadings typical of biodiesel wastewater and/or by increasing the buffering capacity of the anode medium. In one exemplary embodiment, glycerol consumption is increased by up to 50 g/L and ethanol production occurs at rates of up to 10 g/L, values which greatly exceed the capacity of the anode biofilms to concomitantly remove the fermentation byproducts. As a result, in one embodiment 1,3-propanediol can be generated as a metabolic sink for electrons not converted into electricity syntrophically.

Therefore, in addition to producing primarily ethanol as a bioproduct, in various embodiments, the system described herein can be configured to produce primarily 1,3-propanediol (PDO). In one embodiment, both PDO and ethanol are produced in various quantities. PDO is a valuable precursor to a formulation of polyester (polypropylene terephthalate) and for the synthesis of biodegradable plastics. It is also possible that other products, such as other alcohols and diols can also be produced using the MECs and methods described herein.

Embodiments of the invention will be further described by reference to the following examples, which are offered to further illustrate various embodiments of the present invention. It should be understood, however, that many variations and modifications may be made while remaining within the scope of the present invention.

### Examples

Methods—The examples below show embodiments of the typical H-type two chamber MECs as shown in FIG. 3A and the SCMEC as shown in FIG. 3C. SCMEC reactors simplify operation and permit scaling up.

The electricigenic strain GR52 of G. sulfurreducens was pre-grown in DB medium as described in. Speers et al. Appl. Environ Microbiol. 78, 437-444, (2012) with acetate and lactate as electron donors and fumarate as electron acceptor and the cells were harvested by centrifugation and inoculated into the reactor, as previously described in Awate et al. (2017).

Water samples to be treated were supplemented with a mix of minerals, salts and vitamins (as in the DB medium) as described in Speers et al. Appl. Environ Microbiol. 78, 437-444, (2012) to provide optimal nutritional requirements for the growth of the electricigens on the anode electrode. NH₄Cl (0.2 g/L) and bicarbonate was added to provide a nitrogen source and a buffering system when CO₂ is present in the reactor's headspace. The suitability of this supplementation was demonstrated to provide the necessary nutrients for the pre-seeding of anode electrodes treating the process condensate wastewater that is recycled back in a bioethanol refinery. The process condensate wastewater carries fermentation inhibitors (acetate and lactate) that strain GR52 can efficiently oxidize in BES. Supplementation with the other nutrients, generates a medium (Condensate Medium or CM) suitable for pre-seeding of the anode electrode in BES.

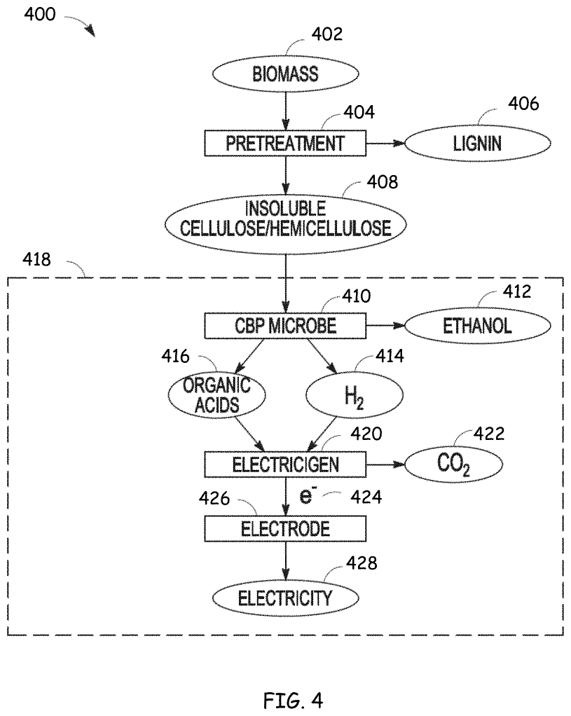

Example 1—The suitability of supplementation of wastewater to provide the necessary nutrients for the pre-seeding of anode electrodes was demonstrated. The process condensate wastewater that is recycled back in a bioethanol refinery was treated. The process condensate wastewater carried fermentation inhibitors (acetate and lactate) that strain GR52 can efficiently oxidize in BES. Supplementation with the other nutrients, generates a medium (Condensate Medium or CM) suitable for pre-seeding of the anode electrode in a BES. FIGS. 5A and 5B show H-type two-chambered MECs run using standard DB medium with 0.5 mM lactate-10 mM acetate versus CM medium (temperature 35° C.), which promote rapid initiation of current generation (in mA) as the cells attached to and grow on the electrode more rapidly. The two lines in FIGS. 5A and 5B are two independent runs to show technical biological variability. In FIG. 5A, the current density generated by electrodes functionalized in DBAL medium in two independent runs is about 83.3 and about 167 uA/cm² reached in about 5 hours. Furthermore, CM-grown bioelectrodes were more efficient, as indicated by the higher yields of electrical current that are reached in these BES as shown in FIG. 5B. In FIG. 5B, the current density generated by electrodes functionalized in CM medium in two independent runs is about 250 and about 500 uA/cm² reached in 5 hours.

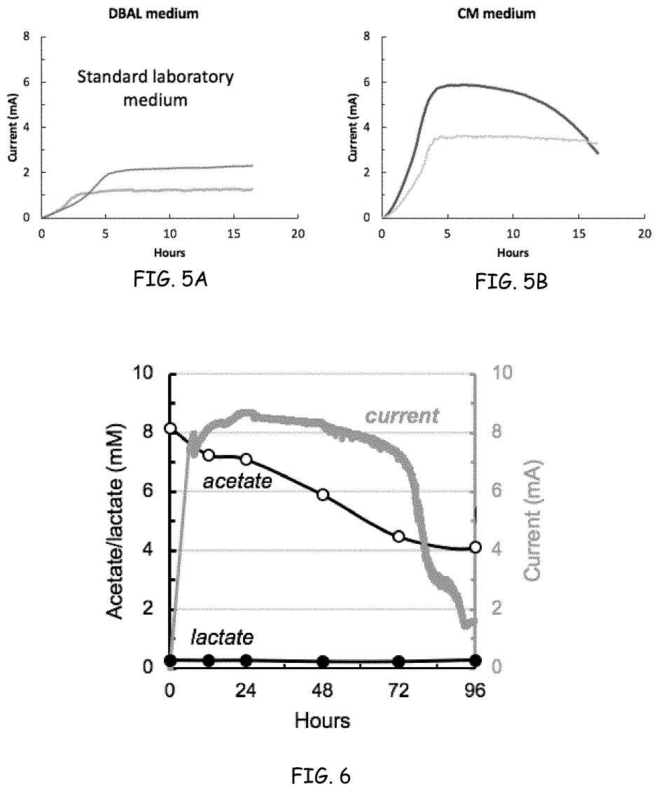

Example 2—The suitability of the method to pre-seed the anode electrodes of 2-L SCMEC reactors was demonstrated. For this experiment, 1L of non-sterile CM was added and the headspace with CO₂ was sparged to buffer the media at neutral pH. Current production was initiated by the addition of GR52 cells harvested from 500 ml of DB-AL pre-cultures and reached a plateau at ˜7 h, indicative that the electricigenic biofilms was fully established on the anode electrode in such short period of time. FIG. 6 shows the current production and acetate/lactate removal in a 2-L SCMEC driven by strain GR52 in CM medium (temperature 35° C.).

All of the lactate was removed as soon as the cells were added to the reactors and current production was coupled to the removal of acetate for 72 h.

Importantly, there was not contamination in the reactors although the CM medium had not been autoclaved.

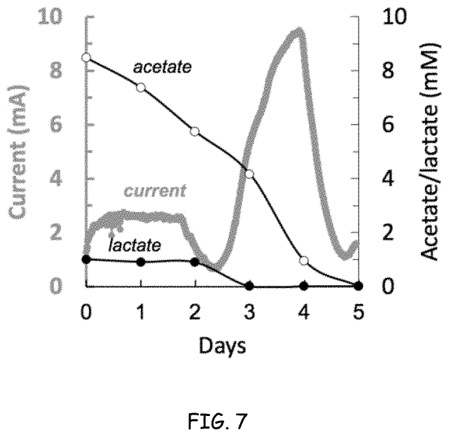

Example 3—The pre-seeding method can be applied to establish a hybrid electrode to control the fermentation products that can be removed by the bioelectrode. The anode electrode of a 2-L SCMEC was pre-seeded with a 50:50 mix of strain GR52 (efficient lactate oxidizer) and the ancestor strain 51 (efficient acetate oxidizer). For these experiments, the Condensate medium (CM) was modified to include ten-times the amount of the nitrogen source (2 g/L NH₄Cl) and the bicarbonate was removed to control for the pH externally via the additions of a NaOH. FIG. 7 shows the current production and acetate/lactate removal in a 2-L SCMEC driven by a hybrid GR52:GR51 bioelectrode in CM medium (temperature 35° C.). As shown in FIG. 3, the hybrid bioelectrode generated current in a stepwise fashion matching the removal of acetate first and then lactate and acetate. By day 4, 95% of the acids had been consumed.

Thus, it is possible to custom-tailor the activities of the electricigenic cells in the bioelectrode by controlling the type and concentration of cells used for pre-seeding. Furthermore, operational parameters such as the anode potential used to establish the electricigenic biofilm and media conditions can be adjusted during pre-seeding to modulate the growth of the electricigenic cells and their activities for specific applications.

Embodiments described herein provide a method of making an electrode. The improved method allows for functionalization of an electrode for transfer of electrons between the electricigenic biofilm and the electrode. The electrode can be functionalized in less than 24 hours by generating increased levels of current. The method allows for an electricigenic biofilm to be formed on the electrode surface with high concentrations of electricigenic bacteria. The method also allows for the inoculation of a second microorganism, such as a fermentative bacterium to develop a microbial consortium for the metabolism of complex substrates such as sugars and sugar alcohols into CO₂ and/or co-products (e.g., ethanol, PDO) and the simultaneous generation of electricity. The method also discourages the spontaneous growth of fastidious organisms present in the liquid increasing the coulombic efficiency of the electrode. The electrode is functionalized without the need for sterile conditions or autoclaved media leading to considerable cost savings.

In one embodiment, a novel method for making electrodes is described herein. The method can functionalize electrodes for transfer of electrons between electricigenic biofilm on the electrode surface and the electrode. The method can include poising the electrode at a potential for metabolism of one or more selected substrates by one or more electricigenic bacteria, wherein the electrode is located in a chamber with a liquid. In one embodiment, the metabolism is oxidative metabolism.

In one embodiment, the method includes pre-seeding the electrode in the chamber with an inoculum of electricigenic bacteria, wherein the electricigenic bacteria in the inoculum have been grown in one or more growth mediums comprising said selected substrates.

In one embodiment, the method includes allowing the electricigenic bacteria to attach to the electrode to form an electricigenic biofilm on the electrode surface, wherein the electrode is functionalized in less than 24 hours.

In one embodiment, the method includes allowing a second organism to attach to the electricigenic biofilm and couple their metabolic activities to expand the catalytic power of the electricigenic biofilm.

In one embodiment, the method includes functionalizing the electrode in less than 12 hours.

In one embodiment, the electricigenic biofilm has a thickness of at least 0.5 μm.

In one embodiment, the electrode generates a current density of greater than about 50 μA per cm² in less than 24 hours.

In one embodiment, the biofilm comprises two or more strains of electricigenic bacteria.

In one embodiment, the method minimizes growth of fastidious organisms on the electrode.

In one embodiment, the substrates are selected from the group consisting of acetate, lactate, fumarate, and combinations thereof.

In one embodiment, the electricigenic biofilm comprises a strain of Geobacter sulfurreducens (Gsu).

In one embodiment, one or more strains of G. sulfurreducens (Gsu) are selected from the group consisting of Gsu, GsulA, GR51, GR52, and combinations thereof.

In one embodiment, the method includes adding said supplements to the liquid in the chamber for growth of the electricigenic bacteria.

In one embodiment, the supplements are selected from the group consisting of nutrients, vitamins, salts, minerals, a nitrogen source, and combinations thereof.

In one embodiment, the functionalization of the electrode occurs in one or more non-autoclaved mediums.

In one embodiment, the one or more growth mediums and/or the liquid in the chamber comprises one or more condensate mediums (CMs).

Embodiments described herein provide a method of treating wastewater with the improved electrodes described herein.

In one embodiment, the method includes treating wastewater comprising providing a first electrode in a chamber with wastewater, wherein the first electrode has been functionalized by a method comprising poising the electrode at a potential optimal for metabolism of one or more selected substrates by one or more electricigenic bacteria, pre-seeding the first electrode with an inoculum of electricigenic bacteria, wherein the electricigenic bacteria in the inoculum have been grown in one or more growth mediums comprising said substrates, wherein said substrates are the metabolic targets by the electricigenic bacteria in the wastewater, and allowing the electricigenic bacteria to attach to the electrode to form an electricigenic biofilm. In one embodiment, the metabolism is oxidative metabolism carried out by the electricigenic bacteria.

In one embodiment, the method includes adding said supplements to the wastewater in the electrode chamber for growth of the electricigenic bacteria.

In one embodiment, the first electrode is the anode electrode and the second electrode is the cathode electrode.

In one embodiment, the electrodes are placed in a single chamber to form a single chamber BES.

In one embodiment, the electrodes are placed in different chambers to form a dual-chamber cell BES.

In one embodiment, the electrodes are configured to form a microbial fuel cell.

In one embodiment, the electrodes are configured to form a microbial electrolysis cell (MEC).

Embodiments described herein provide an improved electrode. In one embodiment, a functionalized electrode comprising an electricigenic film having a thickness of about 0.5 μm is provided wherein the electrode generates a current density of greater than about 50 μA per cm2 in less than 24 hours. In other embodiments, a functionalized electrode comprising an electricigenic film of less than 10 μm thickness is provided wherein the electrode generates a current density of greater than about 50 μA per cm² in less than 24 hours.

In one embodiment, the electricigenic biofilm comprises a strain of G. sulfurreducens.

In one embodiment, the strain(s) of Gsu are selected from the group consisting of Gsu, GsulA, GR51, GR52, and combinations thereof.

In one embodiment, the electrode is housed in a microbial fuel cell.

In one embodiment, the electrode is housed in a microbial electrolysis cell.

The various embodiments described herein highlight the potential of improved methods functionalizing electrodes and incorporation of the improved electrodes in BESs. The improved electrodes allow various additional industrial needs to be met.

All publications, patents and patent documents are incorporated by reference herein, as though individually incorporated by reference, each in their entirety, as though individually incorporated by reference. In the case of any inconsistencies, the present disclosure, including any definitions therein, will prevail.

Although specific embodiments have been illustrated and described herein, it will be appreciated by those of ordinary skill in the art that any procedure that is calculated to achieve the same purpose may be substituted for the specific embodiments shown. This application is intended to cover any adaptations or variations of the present subject matter.

What is claimed is:

1. A method of making an electrode comprising:
poising the electrode at a potential for metabolism of one or more selected substrates by one or more electricigenic bacteria, wherein the electrode is located in a chamber with a liquid;
pre-seeding the electrode in the chamber with an inoculum of electricigenic bacteria, wherein the electricigenic bacteria in the inoculum have been grown in one or more growth mediums comprising said selected substrates; and
allowing the electricigenic bacteria to attach to the electrode to form an electricigenic biofilm on the surface of the electrode, wherein the electrode is functionalized for transfer of electrons between the electricigenic biofilm and the electrode in less than 24 hours.

2. The method of claim 1, wherein the metabolism is oxidative metabolism.

3. The method of claim 1, wherein the electrode is functionalized for generation of current in less than 12 hours.

4. The method of claim 1, wherein the electricigenic biofilm has a thickness of at least 0.5 μm.

5. The method of claim 1, wherein the electrode generates a current density of greater than 50 μA per cm² in less than 24 hours.

6. The method of claim 1, wherein the method minimizes growth of fastidious organisms on the electrode.

7. The method of claim 1, wherein the substrates are selected from the group consisting of acetate, lactate, pyruvate, and combinations thereof.

8. The method of claim 1, wherein the electricigenic biofilm comprises a strain of Geobacter sulfurreducens (Gsu).

9. The method of claim 1, wherein the strain(s) of Gsu are selected from the group consisting of GsuA, GR51, GR52, and combinations thereof.

10. The method of claim 1, further comprising adding one or more supplements to the liquid in the chamber for growth and attachment of the electricigenic bacteria.

11. The method of claim 1, wherein said supplements are selected from the group consisting of nutrients, vitamins, salts, minerals, a nitrogen source, and combinations thereof.

12. The method of claim 1, wherein the functionalization of the electrode occurs in one or more non-autoclaved mediums.

13. The method of claim 1, wherein the one or more growth mediums and/or the liquid in the chamber comprises one or more condensate mediums (CMs).

14. A method of treating wastewater comprising:
providing a first electrode in a chamber with wastewater, wherein the first electrode has been functionalized by a method comprising poising the electrode at a potential for metabolism of one or more selected substrates by one or more electricigenic bacteria;
pre-seeding the first electrode with an inoculum of electricigenic bacteria, wherein the electricigenic bacteria in the inoculum have been grown in one or more growth mediums comprising said substrates, wherein said substrates are the metabolic targets by the electricigenic bacteria in the wastewater and allowing the electricigenic bacteria to attach to the electrode to form an electricigenic biofilm;
providing a second electrode; and providing a reference electrode.

15. The method of claim 14, wherein the first electrode is the anode electrode and the second electrode is the cathode electrode.

16. The method of claim 14, wherein the electrodes are placed in a single chamber to form a single chamber BES.

17. The method of claim 14, wherein the electrodes are placed in different chambers to form a dual-chamber cell BES.

18. The method of claim 14, wherein the electrodes are configured to form a microbial fuel cell.

19. The method of claim 14, wherein the electrodes are configured to form a microbial electrolysis cell.

20. The method of claim 14, wherein the method of functionalizing the electrode further comprises adding one or more supplements to the wastewater for growth and attachment of the electricigenic bacteria.

21. A functionalized electrode comprising an electricigenic film having a thickness of at least about 0.5 μm, wherein the electrode generates a current density of greater than about 50 μA per cm² in less than 24 hours.

22. The electrode of claim 21, wherein the electricigenic biofilm comprises a strain of G. sulfurreducens.

23. The electrode of claim 21, wherein the strain(s) of GSu are selected from the group consisting of GsulA, GR51, GR52, and combinations thereof.

24. The electrode of claim 21, wherein the electrode is housed in a microbial fuel cell.

25. The electrode of claim 21, wherein the electrode is housed in a microbial electrolysis cell.
